(19) United States
(12) Patent Application Publication
(10) Pub. No.: US 2006/0194262 A1
(43) Pub. Date: Aug. 31, 2006

(54) P27 ubiquitination assay and methods of use

(76) Inventors: Shuichan Xu, San Diego, CA (US); Sarah Cox, San Diego, CA (US); Weilin Xie, San Diego, CA (US); Frank M. Mercurio, San Diego, CA (US)

Correspondence Address: JONES DAY, 222 EAST 41ST ST, NEW YORK, NY, 10017, US

(21) Appl. No.: 11/253,128

(22) Filed: Oct. 17, 2005

Related U.S. Application Data
(60) Provisional application No. 60/619,092, filed on Oct. 15, 2004.

(51) Int. Cl. G01N 33/53

(52) U.S. Cl. 435/7.5

(57) ABSTRACT

This invention relates to easy and reliable assays for the ubiquitination of p27. Because the assay can be performed as a plate capture assay, or as a homogenous time-resolved fluorescence resonance energy transfer assay, with defined, easily replicatable reaction conditions, it is particularly useful for high-throughput screening of, for example, a prospective anticancer agent. The invention provides methods to determine the amount of ubiquitination of p27, or of any protein, and use of the method to identify compounds that modulate the ubiquitination of p27, or any protein.

This application claims benefit of U.S. Provisional Application Ser. No. 60/619,092, filed Oct. 15, 2005, which is incorporated herein by reference in its entirety.

### FIELD OF THE INVENTION

This invention relates to assays for p27 ubiquitination. This invention also encompasses screening methods for the identification of compounds that modulate the ubiquitination of protein p27. Because the degradation of p27 is associated with the ontogeny of several cancers, this invention further provides for the use of the screening method to identify potential anti-cancer compounds.

### BACKGROUND OF THE INVENTION

p27 is a critical negative regulator of cell cycle control that specifically inhibits the transition from G₁ to S phase. In particular, p27 is a negative regulator of the cell cycle proteins Cdk2-cyclin E and Cdk2-cyclin A, the activities of which are required for this transition. In quiescent, nonreplicating cells, p27 levels accumulate. However, as quiescent cells begin to divide, levels of p27 rapidly fall. p27 levels are reduced through degradation of existing protein, and not through transcriptional or translational controls. This degradation is accomplished by ubiquitination followed by proteasome-mediated degradation.

In cells, ubiquitin is activated by E1 (ubiquitin-activating enzyme) and transferred to E2 (ubiquitin-conjugating enzyme), forming a thioester bond between the active cysteine in E2 and the C-terminal carboxyl group of ubiquitin (E2˜Ub). See Hershko & Ciechanover, Annu. Rev. Biochem., 67:425-479 (1998) for a review. p27 is first marked for ubiquitination by Cdk2-cyclin E, which phosphorylates the protein at the T187 residue. Phosphorylated p27 in complex with Cdk2 and Cyc E is a substrate for an E3 ligase known as SCF^{Skp2}, which consists of the proteins Skp1, Cul1 (cullin), Rbx1 (a Roc protein), and the F-box protein Skp2. The E3 ligase binds both phosphorylated p27 and E2˜Ub and facilitates the transfer of ubiquitin from E2 to p27. The components of the ubiquitin ligation cascade have received considerable attention. See Weissman, Nature Reviews, 2:169-178 (2001) for a review. E1 and E2 are well characterized enzymes. There are several species of E2 (at least 25 in mammals), some of which act in preferred pairs with specific E3 enzymes to confer specificity for different target proteins. While the nomenclature for E2 is not standardized across species, investigators in the field have addressed this issue and the skilled artisan can readily identify various E2 proteins, as well as species homologues. Haas et al., FASEB, 11: 1257-1268 (1997).

Unchecked degradation of cellular regulatory components (e.g., p53, β-catenin, p27) has been observed in certain tumors, suggesting the hypothesis that deregulated ubiquitin ligases play a role in this altered degradation. See A. Ciechanover, EMBO J., 17:7151 (1998) for review. Degradation of p27 has been implicated in the onset and progression of a number of cancers, including cancers of the breast (Alkarain et al., J. Mammary Gland Biol. Neoplasia, 9(1):67-80 (2004)), prostate (Vis et al., J. Urol., 164(6):2156-61 (2000)), endometrium, ovary (Sui et al., Gynecol Oncol., 83(1):56-63 (2001)) and skin. Thus, the cellular proteins responsible for the ubiquitination of p27, and the p27 ubiquitination reaction itself, are important targets to be used for the development of anti-cancer drugs.

Several p27 ubiquitination assays have been described in the art, but none are suitable for drug discovery. Wang et al., J. Biol. Chem., 278(34):32390-32396 (2003) describe an assay of p27 ubiquitination activity in which [³⁵S]-labeled p27, phosphorylated by Cdk2/Cyclin E, is combined with E1, cdc34 (an E2 protein); SCF^{Skp2} complex, ubiquitin, methylated ubiquitin, ubiquitin aldehyde, Cks1, and MG132 (a proteasome inhibitor). Results of the ubiquitination reaction were visualized by SDS-PAGE and phosphorimaging. Issakani et al. (U.S. Pat. No. 6,737,244) describe a ubiquitin ligase assay in which ubiquitin tagged with FLAG (an 8-mer peptide) is combined with ATP, E1, E2-Ubch5c (E2), E3-H is ROC1/Cul1; however, the FLAGged ubiquitin is not transferred to p27. Others have described p27 ubiquitination assays that use cell extracts to supply the necessary components of the ubiquitination reaction. See, e.g., Tsvetkov et al., Curr Biol., 9(12):661-4 (1999).

### 3. SUMMARY OF THE INVENTION

The invention provides a method of determining an amount of ubiquitin in ubiquitinated protein, comprising capturing said ubiquitinated protein on a surface to form captured ubiquitinated protein, and detecting the ubiquitin in said captured ubiquitinated protein. In another embodiment, the invention provides a method of determining the level or amount of ubiquitination of a protein, comprising: (a) contacting said protein with a plurality of polypeptides, said plurality of polypeptides together capable of ubiquitinating said protein, to form ubiquitinated protein; (b) capturing said ubiquitinated protein on a surface; and (c) determining the amount of ubiquitin present in said protein captured on said surface, wherein the amount of ubiquitin present in said protein captured on said surface is correlated with the extent of ubiquitination of said p27. In a specific embodiment, said protein is p27. In an specific embodiment, said plurality of polypeptides are each isolated polypeptides. In a more specific embodiment, said plurality of polypeptides comprises E1, E2, E3, Cks1 and ubiquitin. In a more specific embodiment, said E1, E2, E3 or Cks1 is recombinantly produced. In another more specific embodiment, said E1, E2, E3 and Cks1 are purified from a cell extract. In another specific embodiment, said p27 is pre-phosphorylated. In yet another specific embodiment, said ubiquitin is labeled. In a more specific embodiment, said label is biotin. In another more specific embodiment, said labeled ubiquitin is visualized with Europium that is coupled to streptavidin. In another specific embodiment of the method, the determination occurs in a multi-well plate as part of a high-throughput screen.

In another embodiment, the invention provides a method of determining the amount of ubiquitin in ubiquitinated protein, comprising: (a) labeling said protein with a first label; (b) labeling said ubiquitin with a second label; and (c) determining a ratio of said first label to said second label, wherein said ratio is correlated with the amount of ubiquitin in said protein. In a specific embodiment, said protein is p27. In another specific embodiment, said first label and said second label are fluorescent labels. In a more specific embodiment, said determining a ratio comprises detecting fluorescence from said first label to produce a first fluorescence value; detecting fluorescence from said second label to produce a second fluorescence value; and determining the ratio of said first fluorescence value to said second fluorescence value. In another more specific embodiment, said second label emits a detectable fluorescence signal when said first fluorescent label is excited. In a more specific embodiment, said first label and said second label are present on the same ubiquitinated p27. In another specific embodiment, said first label emits a detectable fluorescence signal when said second fluorescent label is excited. In a more specific embodiment, said first label and said second label are present on the same ubiquitinated p27. In another specific embodiment, said first label and said second label are suitable for use in a fluorescence resonance energy transfer assay. In another specific embodiment, said first label is Europium, Cy5, trisbipyridine europium cryptate, Dylight™ 547, Dylight™ 647, or allophycocyanine (XL665). In another specific embodiment, said second label is Europium, Cy5, trisbipyridine europium cryptate, Dylight™ 547, Dylight™ 647, or allophycocyanine (XL665). In another specific embodiment, said first label is Europium and said second label is Cy5. In a more specific embodiment, said determining a ratio comprises exciting said Europium at 340 nm, determining the fluorescence of said Europium at 620 nm to produce a first fluorescence value, determining the fluorescence of said Cy5 at 665 nm to produce a second fluorescence value, and determining a ratio of the second fluorescence value and the first fluorescence value, wherein a higher ratio indicates a greater amount of p27 ubiquitination. In a specific embodiment of the assay, said Eu is present at a concentration of about 2 nM, and the concentration of Cy5 about 125 nM.

In another embodiment, the invention provides a method of determining the amount of ubiquitination of p27, comprising: (a) ubiquitinating p27 with ubiquitin to form ubiquitinated p27, wherein said ubiquitin is labeled with a first label either before or after said ubiquitinating; (b) labeling said p27 with a second label differentiable from said first label; and (c) determining a ratio of a second signal from said second label to a first signal from said first label; wherein a higher ratio indicates a greater amount of p27 ubiquitination. In a specific embodiment, said first label and said second label are fluorescent labels, and said first signal and said second signals are fluorescence signals from said first label and said second label, respectively. In a more specific embodiment, said first fluorescent label and said second fluorescent label are suitable for use in a fluorescence resonance energy transfer or luminescence resonance energy transfer assay. In another specific embodiment, said first fluorescent or luminescent label is a donor and said second fluorescent or luminescent label is an acceptor. In another embodiment, the invention provides a method of determining the amount of ubiquitin bound to a protein, comprising determining a ratio of an amount of a second label on said ubiquitin to an amount of a first label on said protein, wherein the ratio is correlated with the amount of ubiquitination of said protein.

In specific embodiments of the above, said ubiquitinated protein has been formed from a protein by contacting said protein in vitro with a plurality of polypeptides that are together sufficient to ubiquitinate said protein. In a specific embodiment, each polypeptide in said plurality of polypeptides is an isolated polypeptide. In a more specific embodiment, said plurality of polypeptides comprises E1, E2, E3, Cks1 and ubiquitin. In a more specific embodiment, said ubiquitin comprises unlabeled ubiquitin and labeled ubiquitin. In a more specific embodiment, said E1, E2, E3 or Cks1 is recombinantly produced. In another more specific embodiment, said E1, E2, E3 and Cks1 are purified from a cell extract. In another more specific embodiment, said E1 is present at a concentration of about 5 ng/μL; said E2 is present at a concentration of about 150 ng/μL; said E3 is present at a concentration of about 5 ng/μL; and/or said Cks1 is present at a concentration of about 0.25-ng/μL. In another more specific embodiment, said labeled ubiquitin comprises biotin, and is present in a concentration of about 9 ng/μL. In another more specific embodiment, said E1 is present at a concentration of about 5 ng/μL; said E2 is present at a concentration of about 150 ng/μL; said E3 is present at a concentration of about 12.5 ng/μL; and/or said phosphorylated p27 is present at a concentration of about 4 ng/μL.

In another embodiment, the invention provides a method of identifying a compound that modulates ubiquitination of p27, comprising determining the amount of ubiquitinated p27 formed by combining isolated phosphorylated p27, E1, E2, E3, Cks1 and ubiquitin in the presence of said compound and in the absence of said compound, wherein, if the amount of ubiquitinated p27 formed in the presence of said compound differs from the amount of ubiquitinated p27 formed in the absence of said compound, said compound is identified as a compound that modulates the ubiquitination of p27. In a specific embodiment, said p27 is phosphorylated with Cdk2 and Cyclin E prior to combination with said E1, E2, E3, Cdk2 and ubiquitin. In another specific embodiment, said phosphorylated p27 is present at a concentration of about 4 ng/μL; said E1 is present at a concentration of about 5 ng/μL; said E2 is present at a concentration of about 150 ng/μL; said Cks1 is present at a concentration of about 0.25 ng/μL; said ubiquitin is present at a concentration of about 250 ng/μL; or said E3 is present at a concentration of about 5 ng/μL. In another specific embodiment, said amount of ubiquitinated p27 formed in the presence of said compound is lower than the amount of ubiquitinated p27 formed in the absence of said compound, and said compound is identified as a compound that inhibits the ubiquitination of p27. In another specific embodiment, said E1, E2, E3 or Cks1 are recombinantly produced. In another specific embodiment, said E1, E2, E3 or Cks1 are purified from a cell extract. In another specific embodiment, said ubiquitin is labeled with a label. In a more specific embodiment, said label is biotin. In a more specific embodiment, said labeled ubiquitin is visualized using Europium-labeled streptavidin. In another specific embodiment, said identification occurs in a multi-well plate as part of a high-throughput screen.

In another embodiment, the invention provides a method of identifying a compound that modulates ubiquitination of p27 in a sample, comprising: (a) preparing a first sample by combining an amount of p27 with a compound to form a mixture of said p27 and said compound; (b) preparing a second sample comprising same amount of p27, but without said compound; (c) respectively contacting the first and second samples with a plurality of polypeptides, said plurality of polypeptides together capable of ubiquitinating p27, to form ubiquitinated p27; (d) respectively capturing said ubiquitinated p27 in the first and second samples on a surface; and (e) respectively determining, in the first and second samples, the amounts of ubiquitin present in said p27 captured on said surface; wherein the compound modulates ubiquitination if the amounts of ubiquitin obtained from the first and second samples are different. In a specific embodiment of this method, said preparation of said first sample and said second sample additionally comprises combining p27 with E1, E2, E3, Cks1, ubiquitin, or any combination thereof. In another specific example, said plurality of polypeptides are each isolated polypeptides. In a more specific embodiment, said plurality of polypeptides comprises E1, E2, E3, Cks1 and ubiquitin. In a more specific embodiment, said E1, E2, E3 or Cks1 is recombinantly produced. In another more specific embodiment, said E1, E2, E3, and Cks1 are purified from a cell extract. In another more specific embodiment said ubiquitin is labeled. In a more specific embodiment, said label is biotin. In a more specific embodiment, said labeled ubiquitin is visualized using Europium labeled streptavidin. In another specific embodiment of the method, the identification of the compounds that modulated ubiquitination of p27 occurs in a multi-well plate as part of a high-throughput screen.

In another embodiment, the invention provides a method of identifying a compound that modulates the ubiquitination of a protein (e.g., an anticancer agent), comprising: (a) ubiquitinating said protein in the presence and in the absence of said compound; (b) labeling said protein with a first label; (c) labeling said ubiquitin with a second label; and (d) determining a ratio of said first label to said second label, wherein if said ratio in the presence of said compound is different than said ratio in the absence of said compound, said compound modulates the ubiquitination of p27. In a specific embodiment, said protein is p27. In another specific embodiment, said first label and said second label are fluorescent labels. In a more specific embodiment, said determining a ratio comprises detecting fluorescence from said first label to produce a first fluorescence value; detecting fluorescence from said second label to produce a second fluorescence value; and determining the ratio of said first fluorescence value to said second fluorescence value. In another more specific embodiment, said second label emits a detectable fluorescence signal when said first fluorescent label is excited. In a more specific embodiment, said first label and said second label are present on the same ubiquitinated p27. In another specific embodiment, said first label emits a detectable fluorescence signal when said second fluorescent label is excited. In a more specific embodiment, said first label and said second label are present on the same ubiquitinated p27. In another specific embodiment, said first label and said second label are suitable for use in a fluorescence resonance energy transfer assay. In another specific embodiment, said first label is Europium, Cy5, trisbipyridine europium cryptate, Dylight™ 547, Dylight™ 647, or allophycocyanine (XL665). In another specific embodiment, said second label is Europium, Cy5, trisbipyridine europium cryptate, Dylight™ 547, Dylight™ 647, or allophycocyanine (XL665). In another specific embodiment, said first label is Europium and said second label is Cy5. In another specific embodiment, said second label is Europium and first second label is Cy5. In a more specific embodiment, said Europium is excited by radiation at 340 nm, the fluorescence of said Europium at 620 nm is determined to produce a first fluorescence value, the fluorescence of said Cy5 at 665 nm is determined to produce a second fluorescence value, and a ratio of the second fluorescence value to the first fluorescence value is determined, wherein a higher ratio indicates a greater amount of p27 ubiquitination.

The invention further provides a method of identifying an anticancer agent, comprising determining the amount of ubiquitinated p27 formed by combining isolated p27, E1, E2, E3, Cks1, Cyclin E, Cdk2 and ubiquitin in the presence of said compound and in the absence of said compound; wherein, if the amount of ubiquitinated p27 formed in the presence of said compound differs from the amount of ubiquitinated p27 formed in the absence of said compound, said compound is identified as an anticancer agent. In a specific embodiment, said p27 is phosphorylated with Cdk2 and Cyclin E prior to combination with said E1, E2, E3, Cdk2 and ubiquitin. In another specific embodiment, said phosphorylated p27 is present at a concentration of about 4 ng/μL; said E1 is present at a concentration of about 5 ng/μL; said E2 is present at a concentration of about 150 ng/μL; or said E3 is present at a concentration of about 5 ng/μL. In another specific embodiment, said amount of ubiquitinated p27 formed in the presence of said compound is lower the amount of ubiquitinated p27 formed in the absence of said compound. In another specific embodiment, said E1, E2, E3 or Cks1 are recombinantly produced. In another specific embodiment, said E1, E2, E3, and Cks1 are purified from a cell extract. In another specific embodiment, said ubiquitin is labeled. In a more specific embodiment, said label is biotin. In a more specific embodiment, said labeled ubiquitin is visualized using Europium labeled streptavidin. In another specific embodiment, said identification occurs in a multi-well plate as part of a high-throughput screen.

The invention also provides a method of identifying an anticancer agent comprising: (a) preparing a first sample by combining an amount of p27 with a compound to form a mixture of said p27 and said compound; (b) preparing a second sample comprising same amount of p27, but without the compound; (c) respectively contacting the first and second samples with a plurality of polypeptides, said plurality of polypeptides together capable of ubiquitinating p27, to form ubiquitinated p27; (d) respectively capturing said ubiquitinated p27 in the first and second samples on a surface; and (e) respectively determining, in the first and second samples, the amounts of ubiquitin present in p27 captured on said surface; wherein the compound is an anticancer agent if the amounts of ubiquitin obtained from the first sample is lower than the amount obtained from the second sample. In a specific embodiment of this method, said preparation of said first sample and said second sample additionally comprises combining p27 with E1, E2, E3, Cks1, ubiquitin, or any combination thereof. In another specific example of this method, said plurality of polypeptides are each isolated polypeptides. In a more specific embodiment, said plurality of polypeptides comprises E1, E2, E3, Cks1 and ubiquitin. In a more specific embodiment, said E1, E2, E3 or Cks1 is recombinantly produced. In another more specific embodiment, said E1, E2, E3, and Cks1 are purified from a cell extract. In another more specific embodiment, said ubiquitin is labeled. In a more specific embodiment, said label is biotin. In an even more specific embodiment, said labeled ubiquitin is visualized using Europium labeled streptavidin. In another specific embodiment of the method, the identification occurs in a multi-well plate as part of a high-throughput screen.

The invention also provides a method of identifying an anticancer agent comprising determining whether a compound changes the level or amount of ubiquitination of p27 as determined by a change in the ratio of a first and second label, or signal from a first and second label, wherein said p27 is labeled with said first label and ubiquitin is labeled with said second label, wherein if a difference exits, the compound is identified as an anticancer agent. In another embodiment, the invention provides a method of identifying an anticancer agent comprising (a) preparing a first sample by combining an amount of p27 with a compound to form a mixture of said p27 and said compound; (b) preparing a second sample comprising same amount of p27, but without the compound; (c) respectively contacting the first and second samples with a plurality of polypeptides comprising ubiquitin, said plurality of polypeptides together capable of ubiquitinating p27, to form first and second ubiquitinated p27, respectively; (d) labeling said p27 in said first and second ubiquitinated p27 with a first label and said ubiquitin in said first and second ubiquitinated p27 with a second label; and (e) respectively determining, for said first and second ubiquitinated p27, a ratio of an amount of, or a ratio of an amount of signal from, said first label to said second label; wherein the compound is an anticancer agent if said ratio is different for said second ubiquitinated p27 from said first ubiquitinated p27. In a specific embodiment of this method, said preparation of said first sample and said second sample additionally comprises combining p27 with E1, E2, E3, Cks1, ubiquitin, or any combination thereof. In another specific example of this method, said plurality of polypeptides are each isolated polypeptides. In a more specific embodiment, said plurality of polypeptides comprises E1, E2, E3, Cks1 and ubiquitin. In a more specific embodiment, said E1, E2, E3 or Cks1 is recombinantly produced. In another more specific embodiment, said E1, E2, E3, and Cks1 are purified from a cell extract. In another specific embodiment of the method, the identification occurs in a multi-well plate as part of a high-throughput screen. In a specific embodiment, said p27 is phosphorylated with Cdk2 and Cyclin E prior to combination with said E1, E2, E3, Cdk2 and ubiquitin. In another specific embodiment, said phosphorylated p27 is present at a concentration of about 4 ng/μL; said. E1 is present at a concentration of about 5 ng/μL; said E2 is present at a concentration of about 150 ng/μL; or said E3 is present at a concentration of about 12.5 ng/μL.

The invention further provides a kit for p27 ubiquitination assay comprising p27; Cdk2/Cyclin E; and a plurality of polypeptides, said plurality of polypeptides together capable of ubiquitinating p27, recombinant cells expressing the polypeptides, or recombinant vectors harboring sequences which encode the polypeptides. In a specific embodiment of the kit, p27 and Cdk2/Cyclin E are provided as a complex. In another specific embodiment, said plurality of polypeptides comprises E1, E2, E3, Cks1 and ubiquitin. In a more specific embodiment, said ubiquitin is labeled. In a more specific embodiment of the kit, said label is biotin. In another more specific embodiment, the kit further comprises one or more of: plates, optionally coated with Protein A or G; buffers; visualization reagents; and apparatus and reagents required for expression and purification of the polypeptides.

The invention also provides a method of identifying a compound that modulates ubiquitination of p27 in a sample, comprising: a. preparing a first sample by combining p27, E1, E2, E3, Cks1, ubiquitin, or any combination thereof with a compound to form a mixture of said compound and said p27, E1, E2, E3, Cks1, ubiquitin, or any combination thereof; b. preparing a second sample by combining p27, E1, E2, E3, Cks1, ubiquitin, or any combination thereof to form a mixture of said p27, E1, E2, E3, Cks1, ubiquitin, or any combination thereof, wherein said second sample lacks said compound; c. optionally, where said mixture in steps (a) and (b) of said p27, E1, E2, E3, Cks1, ubiquitin, or any combination thereof is insufficient to ubiquitinate p27, contacting each of said first and second samples with a plurality of polypeptides, said plurality of polypeptides and said mixture of said p27, E1, E2, E3, Cks1, ubiquitin, or any combination thereof together capable of ubiquitinating p27; d. forming ubiquitinated p27; e. respectively capturing said ubiquitinated p27 in the first and second samples on a surface; and f. respectively determining, in the first and second samples, the amounts of ubiquitin present in said p27 captured on said surface; wherein the compound is identified as a compound that modulates ubiquitination of p27 if the amounts of ubiquitin obtained from the first and second samples are different. The invention also provides a method of identifying an anticancer compound, comprising: a. preparing a first sample by combining p27, E1, E2, E3, Cks1, ubiquitin, or any combination thereof with a compound to form a mixture of said compound and said p27, E1, E2, E3, Cks1, ubiquitin, or any combination thereof; b. preparing a second sample by combining p27, E1, E2, E3, Cks1, ubiquitin, or any combination thereof to form a mixture of said p27, E1, E2, E3, Cks1, ubiquitin, or any combination thereof, wherein said second sample lacks said compound; c. optionally, where said mixture in steps (a) and (b) of said p27, E1, E2, E3, Cks1 and ubiquitin, or any combination thereof is insufficient to ubiquitinate p27, contacting each of said first and second samples with a plurality of polypeptides, said plurality of polypeptides and said mixture of said p27, E1, E2, E3, Cks1, ubiquitin, or any combination thereof together capable of ubiquitinating p27; d. forming ubiquitinated p27; e. respectively capturing said ubiquitinated p27 in the first and second samples on a surface; and f. respectively determining, in the first and second samples, the amounts of ubiquitin present in said p27 captured on said surface; wherein the compound is identified as an anticancer compound if the amounts of ubiquitin obtained from the first and second samples are different. The invention also provides a method of determining the amount of ubiquitination of p27, comprising: a. ubiquitinating p27 with ubiquitin to form ubiquitinated p27, wherein said ubiquitin is labeled with a first label either before or after said ubiquitinating; b. labeling said p27 with a second fluorescent or luminescent label differentiable from said first fluorescent or luminescent label; and c. determining the ratio of a first fluorescence or luminescence signal from said first fluorescent label to a second fluorescence signal from said second fluorescent label; wherein a higher ratio indicates a greater amount of p27 ubiquitination. In a specific embodiment, said first fluorescent label and said second fluorescent label are suitable for use in a fluorescence resonance energy transfer or luminescence resonance energy transfer assay. In another specific embodiment, said first fluorescent or luminescent label is a donor and said second fluorescent or luminescent label is an acceptor.

3.1 Definitions

As used herein, “p27” means the protein which is an inhibitor (e.g., negative regulator) of Cdk2-cyclin E and Cdk2-cyclin A. As used herein, the term “p27” also encompasses any fragment, portion of, or polypeptide derived from p27, that is, any variant of p27, including tagged p27, that may be useful in the assay described herein. Any fragment, portion of, or polypeptide derived from p27 to be used in the methods described herein must be recognized (i.e., bound) and ubiquitinated by the remaining assay components.

As used herein, “isolated,” for example, in the context of “isolated protein,” means that a particular component of the assay method has been separated from a cell extract or is otherwise not contained within the natural cell milieu from which it is derived. An “isolated” protein or assay component may be combined with other assay components and still be considered isolated.

As used herein, “pre-phosphorylated p27” means p27 that, in complex with Cdk2 and Cyc E, is phosphorylated in the presence of ATP and Mg⁺⁺ by Cdk2 and Cyc E.

As used herein, “E1” means a ubiquitin-activating enzyme.

As used herein, “E2” means a ubiquitin-conjugating enzyme.

As used herein, “E3” means the ubiquitin ligase known as SCF^{Skp2}, which comprises the proteins Skp1, Skp2, Roc1 and cullin (Cul1).

As used herein, “Cks1” means a cell-cycle regulatory protein essential for ubiquitination of p27, and which is an essential activator of SCF^{Skp2}.

As used herein, “label” means a molecule that can be directly (i.e., a primary label) or indirectly (i.e., a secondary label) detected; for example a label can be visualized and/or measured or otherwise identified so that its presence or absence can be known. Persons of skill in the art will recognize that the manner in which this is done depends upon the label. Labels include, but are not limited to, biotin, fluorescent labels, label enzymes and radioisotopes.

As used herein, “ubiquitin” encompasses the full-length protein or fragments or variants thereof, as described elsewhere herein, whether labeled or not.

As used herein, “modulate the ubiquitination of p27” means to cause a detectable change in the level or degree of ubiquitination of p27. “Modulate” encompasses an increase or decrease in the level or amount of ubiquitination of p27, e.g., the number of ubiquitin molecules attached to p27 at a given time, or the rate at which such attachment takes place. “Modulate” also encompasses the complete inhibition of ubiquitination of p27. Preferably, a compound identified by the assay as modulating the ubiquitination of p27 reduces the amount or level of p27 ubiquitination.

As used herein, “polypeptide” means both an entire functional protein (e.g., E1, E2, p27, etc.) or a fragment of any thereof that retains all, or a usable part of, the activity of the entire protein.

### 4. BRIEF DESCRIPTION OF THE FIGURES

FIG. 1 illustrates the interactions between the components used in the p27 ubiquitin assay described herein. p27, complexed with Cdk2 and Cyclin E (Cyc E), is phosphorylated. Contact between the p27 complex and E3 (i.e., SCF^{Skp2} (Cullin (Cul1), Skp 1, Skp2 and Roc)) in the presence of Cks1 allows transfer of ubiquitin (black circle) from E2˜Ub to E2 to p27. The formation of E2˜Ub is catalyzed by E1. p27 may be, as the diagram indicates, multiply-ubiquitinated in the assay.

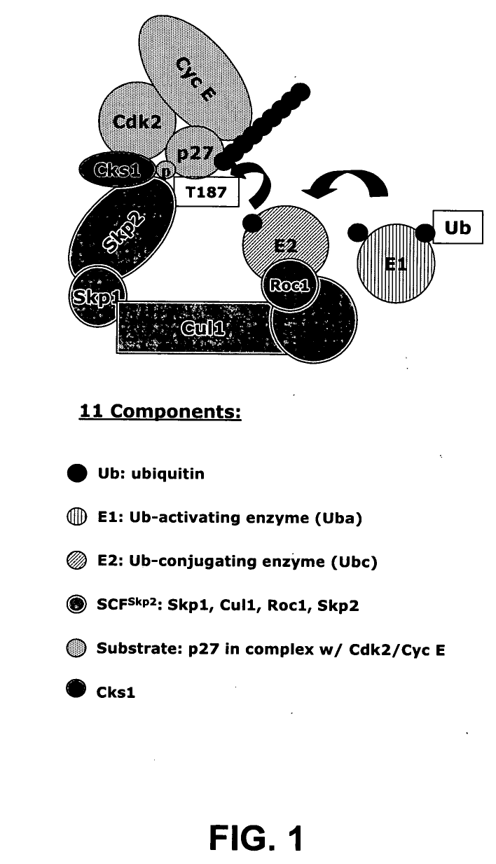

FIG. 2 depicts the results of the assay specificity experiment. A p27 ubiquitination assay (Complete) was performed in the presence of all the components as listed in Table 2 (see Example 2, below). In each control, a single component was omitted from the assay as indicated. Ubiquitinated p27 (4 ng/μl) was captured on Protein A or Protein G plates coated with 2.5 μg/ml of antibody sc-528 and detected with Europium-Streptavidin (1:1000). The value on the Y axis represents p27 ubiquitination as measured by time-resolved fluorescence emission of Europium chelate (excitation at 340 nm and emission at 615 nm).

FIG. 3 depicts the titration of the capturing antibody sc-528 during assay optimization. Two different lots of sc-528 were diluted serially and coated on a protein A-coated 384 well plate. Pre-phosphorylated p27 (4 ng/μl) was completely ubiquitinated and captured on the plate and detected with Eu-Strep (1:1000). The value on the Y axis represents p27 ubiquitination as measured by time-resolved fluorescence emission of Europium chelate (excitation at 340 nm and emission at 615 nm).

FIG. 4 depicts the titration of Europium-Streptavidin during assay optimization. Pre-phosphorylated p27 (4 ng/μl) was completely ubiquitinated and captured on a Protein A plate coated with 2.5 μg/ml of sc-528. Serially diluted Eu-Strep (0.1 mg/ml) was used to detect ubiquitinated p27.

FIG. 5 depicts titration of ubiquitinated p27 during assay optimization. Pre-phosphorylated p27 (8 ng/μl) was completely ubiquitinated, serially diluted in assay diluent, and captured on a Protein A plate coated with 2.5 μg/ml of sc-528. Eu-Strep (0.1 mg/ml) diluted at 1:250 was used to detect ubiquitinated p27.

FIGS. 6A-6C depict the titration of assay components E1 and E2. Pre-phosphorylated p27 was ubiquitinated with either E1 (FIG. 6A) or E2 (FIG. 6B) varied at the concentrations indicated. Other components were held at constant concentrations as indicated in FIG. 6C. p27 was captured on a protein A plate coated with sc-528 (2.5 μg/ml). Ubiquitinated p27 was detected by Eu-streptavidin at a dilution of 1:250.

FIGS. 6D-6F depict titration of assay components E3 and Cks1. Pre-phosphorylated p27 was ubiquitinated with either E3 (FIG. 6D) or Cks1 (FIG. 6E) varied at the concentrations indicated. Other components were held at constant concentrations as indicated in FIG. 6G. p27 was captured on a protein A plate coated with sc-528 (2.5 μg/ml). Ubiquitinated p27 was detected by Eu-streptavidin at a dilution of 1:250.

FIGS. 6G-6I depict titration of E3:Cks1 mixture. Pre-phosphorylated p27 was ubiquitinated with all the components at constant concentrations as indicated in FIG. 6I, except E3 (FIG. 6G) and Cks1 (FIG. 6H). E3 and Cks1 were mixed at a 1:1 molar ratio and diluted serially. Therefore, E3 and Cks1 were titrated at a molar ratio of 1:1 together in this experiment. p27 was captured on a protein A plate coated with sc-528 (2.5 μg/ml). Ubiquitinated p27 was detected by Eu-streptavidin at a dilution of 1:250. The data were plotted against both E3 and Cks1 concentrations.

FIGS. 6J-6L depicts titration of p27 and Bio-Ub. Pre-phosphorylated p27 was ubiquitinated with either p27 or Bio-Ub were varied at the concentrations indicated. Other components were held at constant concentrations as indicated in Table 2. p27 was captured on a protein A plate coated with sc-528 (2.5 μg/ml). Ubiquitinated p27 was detected by Eu-streptavidin at a dilution of 1:250.

FIGS. 7A-7C depicts titration of ATP and dithiothreitol (DTT) during assay optimization. Pre-phosphorylated p27 was ubiquitinated with either DTT (FIG. 7A) or ATP (FIG. 7B) were varied at the concentrations indicated and other components at constant concentrations as indicated in FIG. 7C.

FIGS. 8A-8C depict titration of dimethylsulfoxide (DMSO) during assay optimization. Pre-phosphorylated p27 was ubiquitinated in the presence of increasing amounts of DMSO with all the components at constant concentrations as indicated in FIG. 8C.

FIG. 9A depicts a time course experiment to determine the optimal time to allow p27 ubiquitination to proceed. Pre-phosphorylated p27 was ubiquitinated at room temperature for the indicated times in the presence of all the components listed in FIG. 9B. p27 was captured on a protein A plate coated with sc-528 (2.5 μg/ml). Ubiquitinated p27 was detected by Eu-streptavidin at a dilution of 1:250.

FIG. 10 depicts the results of reaction-stopping optimization. For controls (with and without Cks1), Pre-phosphorylated p27 was ubiquitinated at room temperature with or without Cks1. At the indicated times, an aliquot of the reaction (30 μl) was mixed with 40 μl of Assay Diluent (AD) and frozen on dry ice until all the time points were collected. For AD+/−Cks1, the assay reactions with or without Cks1 were incubated at room temperature. At 15 minutes, 200 μl of AD was added to 150 μl of assay reaction and incubation of the mixture at RT was continued. At indicated time, an aliquot of the mixture (70 μl) was removed and frozen on dry ice until all the time points were collected. p27 was captured on a protein A plate coated with sc-528. Ubiquitinated p27 was detected by Eu-streptavidin.

FIG. 11 depicts the HTR-FRET assay format for p27 ubiquitination. 11A: p27 ubiquitination reaction and reaction components. 11B: detection of ubiquitinated p27. Eu-Europium. Europium was excited by radiation at 340 nm, and fluorescence of europium was read at 620 nm and fluorescence of Cy5 was read at 665 nm. The formula presented for the calculation of FRET values (that is, 665/620*10,000) can be generalized to other donor/acceptor pairs as (acceptor fluorescence value)/(donor fluorescence value)*constant, where donor and acceptor fluorescence values are fluorescence values at a wavelength at which donor or acceptor fluoresce acceptably for detection.

FIG. 12. Determination of the optimal concentration of biotin-labeled ubiquitin. p27 was ubiquitinated at room temperature for 90 min with the indicated concentration of Bio-Ub in the presence or absence of E1. The final concentration of each component in the assay was as follows: His-E1: 5 ng/ml; His-Ubc3:150 ng/ml; E3:10 ng/ml; Cks1: 0.5 ng/ml; and pre-phosphorylated p27: 8 ng/ml. The reaction was stopped by 1:4 dilution with detection mixture containing anti-p27 (sc-527), Eu-PRG (europium-protein G), and SA-XL665 at a final concentration of 10 nM, 5 nM, and 25-125 nM, respectively. The data were collected on Analyst HT (Molecular Devices). Fluorescence of labels was determined at 620 nm and 665 nm. For each of the concentration conditions, the bars in the graph, read from left to right, correspond to the legend, read top to bottom.

FIG. 13. Determination of the optimal concentration of biotin-labeled ubiquitin. p27 was ubiquitinated at room temperature for 1 h with indicated concentrations of Bio-Ub and Ub. The final concentration of each component in the assay is as follows: His-E1: 5 ng/μL; His-Ubc3: 150 ng/μL; E3: 10 ng/μL; Cks1: 0.5 ng/μL; and pre-phosphorylated p27: 8 ng/μL. The reaction was stopped by 1:2 dilution with 2×SDS-loading buffer and subjected to SDS-PAGE and western blotting with anti-p27 and anti-ubiquitin as indicated. Ub: ubiquitin; Bio-Ub: biotin-labeled ubiquitin. Numbers to left of blots are molecular weights in kilodaltons.

FIG. 14. Titration of streptavidin-bound Cy5 (FIG. 14A) and protein G-Europium (FIG. 14B). p27 was ubiquitinated with components listed in the table at room temperature for 1 h in the presence (positive control, S) or absence of E1 (negative control, B). 15 μl of each reaction was stopped with 5 μl of 80 mM EDTA and 20 nM of anti-phospho p27, 5 μl of diluted Cy5-SA, and 5 μl of Eu-PRG in a 384 well plate. The final concentrations for assay components were as follows: His-E1: 5 ng/μL; His-Ubc3: 150 ng/μL; E3: 5 ng/μL; Cks1: 0.25 ng/μL; pre-phosphorylated p27: 4 ng/μL; Bio-Ub: 9.1 ng/μL; ubiquitin: 34.3 ng/μL; DMSO: 2%. EDTA and anti-phosphorylated p27 were 20 mM and 3.33 nM, respectively. The final concentrations of Cy5-SA and Eu-PRG were as indicated in 14A and 14B, respectively. After incubation at room temperature for 1 h, the plate was read on Analyst HT. S/B=(FRET of the sample with E1)/(FRET of the corresponding sample without E1).

FIG. 15. Titration of anti-p27 and protein G-Eu. p27 was ubiquitinated with components listed in the table at room temperature for 1 h in the presence (positive control, S) or absence (negative control, B) of E1. 15 μl of each reaction was stopped with 5 μl of 80 mM EDTA and 750 nM of Cy5-SA, 5 μl of diluted anti-phosphorylated p27, and 5 μl of Eu-PRG in a 384 well plate. The final concentrations for EDTA and Cy5-SA were 20 mM and 125 nM, respectively. The final concentrations of anti-phosphorylated p27 and Eu-PRG were as indicated. The final concentrations for other assay components were as follows: His-E1: 5 ng/μL; His-Ubc3: 150 ng/μL; E3: 5 ng/μL; Cks1: 0.25 ng/μL; pre-phosphorylated p27: 4 ng/μL; Bio-Ub: 9.1 ng/μL; ubiquitin: 34.3 ng/μL; DMSO: 2%. After incubation at RT for 1 h, the plate was read on Analyst HT. S/B=(FRET of the sample with E1)/(FRET of the corresponding sample without E1).

FIG. 16. Titration of E1 and E2. p27 was ubiquitinated with increasing concentrations of E1 or E2 at RT for 1 h in a 384 well plate. Each reaction (15 μl) were incubated with 15 μl of a stopping solution containing 40 mM EDTA, 250 nM of Cy5-SA, 8 nM anti-phosphorylated p27, and 2.5 nM Eu-PRG. The final concentrations for other assay components were as follows: E3: 5 ng/μL; Cks1: 0.25 ng/μL; pre-phosphorylated p27: 4 ng/μL; Bio-Ub: 9.1 ng/μL; ubiquitin: 34.3 ng/μL; DMSO: 2%. After incubation at RT for 1 h, the plate was read on Analyst HT.

FIG. 17. Titration of E3/Cks1 and time course. p27 was ubiquitinated with increasing concentrations of E3 and Cks1 at RT for indicated time in a 384 well plate. E3 was at an equal molar ratio to Cks1 at each dilution. At each time point, 5 μl of 80 mM EDTA was added to 15 ul of reaction. After all the time points were completed, 10 μl of antibody mixture containing 375 nM of Cy5-SA, 12 nM anti-phosphorylated p27, and 3.75 nM Eu-PRG was added to the 20 μL reaction plus EDTA mixture. The final concentrations for other assay components were as follows: His-E1: 5 ng/μL; His-Ubc3: 150 ng/μL; E3: 12.5 ng/μL; Cks1: 0.625 ng/μL; pre-phosphorylated p27: 4 ng/μL; Bio-Ub: 9.1 ng/μL; ubiquitin: 34.3 ng/μL; DMSO: 2%. After incubation at RT for 1 h, the plate was read on Analyst HT.

### 5. DETAILED DESCRIPTION OF THE INVENTION

5.1 p27 Ubiquitination Assay Method

The invention provides methods of assaying the amount or level of ubiquitination of p27. The methods employ an in vitro p27 ubiquitination system reconstituted from individual components. An advantage of the methods described herein is that they are suitable for high-throughput analysis of p27 ubiquitination. When the assays include a test compound (that is, when p27 is phosphorylated or ubiquitinated in the presence of a test compound), the p27 ubiquitination assays described herein enable one to determine whether the compound modulates (e.g., detectably decreases or increases) the amount of ubiquitination of p27. Such compounds are candidates for treatments for diseases, disorders or conditions arising from, or associated with, inappropriate or non-normal ubiquitination of p27 (e.g., abnormally high ubiquitination of p27 relative to normal; a level of p27 ubiquitination that results in tumorigenicity of one or more cell types, etc.).

The assays described herein generally use the following components:

TABLE 1
Components used in high-throughput p27 ubiquitination assay.

| Polypeptide components | Other components |
|---|---|
| p27 | Tris-HCl pH 7.5 |
| Ubiquitin (biotinylated) | MgCl₂ |
| Cyclin E | ATP |
| Cdk2 | DTT |
| Cks1 | DMSO |
| E1 | Anti-p27 antibody |
| E2 | Protein-A coated plates (for plate capture |
| | format) |
| E3 (SCF^{Skp2}): | Enhancement solution |
| Cullin (Cul1) | Eu-streptavidin |
| Skp1 | Assay diluent |
| Skp2 | Coating buffer |
| Roc1 | Wash buffer |
| | Donor fluorophore and acceptor fluorophore |
| | (e.g., protein G-Eu and Bio-Cy5) (for FRET |
| | assay format) |

Specific examples of two formats of the p27 assay are described in the Examples.

5.1.1 Capture Assay

The method of assaying the degree of ubiquitination of p27, in the presence or absence of a compound, can be performed using a plate capture assay in which ubiquitinated p27 is captured to a plate, and the amount of captured ubiquitin is determined.

5.1.1.1 Methods

Generally, the method proceeds as follows. p27 is ubiquitinated and captured to a surface. The ubiquitin in the ubiquitinated p27 is then labeled, and the label is detected. Changes in the amount of label correlated with changes in the level or amount of ubiquitin, and ubiquitination, of p27. In one embodiment, p27 is phosphorylated to make it a suitable substrate for ubiquitination. In one embodiment, p27 is brought into contact with the proteins Cyc E and Cdk2 such that p27 is phosphorylated at, e.g., residue T187, to produce T187-phosphorylated p27. In a specific embodiment, this phosphorylated p27 forms a complex with Cdk2 and Cyc E. In another embodiment, a complex of p27, Cdk2 and Cyc E is contacted with additional Cdk2 and Cyc E to increase the phosphorylation of p27 at T187.

Phosphorylated p27 is then contacted with polypeptides necessary for ubiquitination, in the presence of ATP and MgCl₂. In one embodiment, the polypeptides are E1, E2, E3 (SCF^{Skp2}), Cks1 and a sufficient amount of ubiquitin, to form a p27 ubiquitination reaction mixture. In one embodiment, the ubiquitin is labeled. In a preferred embodiment, the ubiquitin is labeled with biotin. After a period of time, the reaction is stopped, and the ubiquitinated p27 is captured onto a surface. Optionally, a heating step can be included at the end of the reaction. The captured p27 is then assayed to determine the amount of ubiquitin present. Persons of skill in the art will understand that the method steps as outlined above may be varied and still accomplish the goal of the assay.

In one embodiment, the invention provides a method of determining the extent of ubiquitination of p27, comprising contacting p27 with a plurality of polypeptides, said plurality of polypeptides together capable of ubiquitinating p27, to form ubiquitinated p27; capturing said ubiquitinated p27 on a surface; and determining the amount of ubiquitin present in said p27 captured on said surface, wherein the amount of ubiquitin present in said p27 captured on said surface is correlated with the extent of ubiquitination of said p27.

In another embodiment, the invention provides a method of determining the amount of p27 in a sample, comprising contacting p27 with a plurality of polypeptides, said plurality of polypeptides together capable of ubiquitinating p27, to form ubiquitinated p27; capturing said ubiquitinated p27 on a surface; and determining the amount of ubiquitin present in said p27 captured on said surface, wherein the amount of ubiquitin present in said p27 captured on said surface indicates the amount of said p27 in said sample. In a specific example, said sample is derived from a human. In another specific embodiment, said sample is derived from an individual having or suspected of having a disease or condition associated with a deviation of p27 levels from normal. In a more specific embodiment, said disease or condition is cancer.

In another embodiment, the invention provides a method of identifying a compound that modulates ubiquitination of p27, comprising determining the amount of ubiquitinated p27 formed by combining of isolated phosphorylated p27, E1, E2, E3, Cks1 and ubiquitin in the presence of said compound and in the absence of said compound, wherein, if the amount of ubiquitinated p27 formed in the presence of said compound differs from the amount of ubiquitinated p27 formed in the absence of said compound, said compound is identified as a compound that modulates the ubiquitination of p27. In a specific embodiment, said p27 is phosphorylated with Cdk2 and Cyclin E prior to combination with said E1, E2, E3, Cdk2 and ubiquitin. In another specific embodiment, said phosphorylated p27 is present at a concentration of about 4 ng/μL; said E1 is present at a concentration of about 5 ng/μL; said E2 is present at a concentration of about 150 ng/μL; said Cks1 is present at a concentration of about 0.25 ng/μL; said ubiquitin is present at a concentration of about 250 ng/μL; or said E3 is present at a concentration of about 5 ng/μL. In another specific embodiment, said amount of ubiquitinated p27 formed in the presence of said compound is lower the amount of ubiquitinated p27 formed in the absence of said compound, and said compound is identified as a compound that inhibits the ubiquitination of p27. In another specific embodiment, said E1, E2, E3 or Cks1 are recombinantly produced. In another specific embodiment, said E1, E2, E3 or Cks1 are purified from a cell extract. In another specific embodiment, said ubiquitin is labeled with a label. In a more specific embodiment, said label is biotin.

In another embodiment, this invention encompasses a method of identifying a compound that modulates ubiquitination of p27 in a sample comprising: preparing a first sample by combining an amount of p27 with a candidate compound to form a mixture of p27 and the compound; preparing a second sample comprising same amount of p27, but without the compound; respectively contacting the first and second samples with a plurality of polypeptides, said plurality of peptides together capable of ubiquitinating p27, to form ubiquitinated p27; respectively capturing the ubiquitinated p27 in the first and second samples on a surface; and respectively determining the amounts of ubiquitin present in p27 in the first an second samples, captured on the surface, wherein the compound modulates ubiquitination if the amounts of ubiquitination obtained from the first and second samples are different. In a specific embodiment of said method, said first sample and said second sample are independently prepared by combining p27, E1, E2, E3, Cks1, ubiquitin or any combination thereof.

In another embodiment, the method can be used to identify one or more compounds that may be used to treat a disease, disorder or condition associated with the abnormal ubiquitination of p27. In a specific embodiment, said abnormal ubiquitination of p27 is an abnormal increase in ubiquitination. In a more specific embodiment, said abnormal increase in ubiquitination causes, or is associated with, proliferation or metastasis. In another embodiment, the method can be used to identify an anti-cancer agent, where the compound decreases ubiquitination of p27.

In one embodiment of the method, said plurality of polypeptides are each isolated polypeptides, i.e., are not contained within a cell extract or similar substance. In a specific embodiment, said plurality of polypeptides comprises E1, E2, E3, Cks1 and ubiquitin. In a more specific embodiment embodiment, said ubiquitin is labeled. The label may be any detectable label (e.g., colorimetric label, fluorescent, luminescent, radioactive, etc.). In more specific embodiment, said label is biotin. In another embodiment, said p27 or any of said plurality of polypeptides are recombinantly produced. In another embodiment, said p27 or any of said plurality of polypeptides are purified from a cell extract. In another embodiment, said p27 or any of said plurality of polypeptides are individually recombinantly produced and combined in vitro. In another embodiment, two or more of said p27, said Cdk2 and said Cyclin E are co-expressed in a host and purified together. In another embodiment, said E3 consists of the proteins Cul1, Roc1, Skp1 and Skp2, and said Cul1, Roc1, Skp1 and Skp2 are co-expressed in a host and purified together. In another embodiment, said p27 is phosphorylated prior to ubiquitination. In another embodiment, said p27 is phosphorylated by contacting a complex of p27, Cdk2 and cyclin E with a complex of Cdk2 and cyclin E in the presence of ATP and magnesium. In another specific embodiment, said surface is coated with an antibody that binds p27. In a more specific embodiment, said antibody is an antibody that recognizes a plurality of the C-terminal 19 residues of p27.

5.1.1.2 Plates

An essential aspect of the plate capture format of the assay as described herein is the capture of p27 to a surface. This surface is preferably one that is suitable for high-throughput screening of multiple samples, that is, for performing multiple p27 ubiquitination reactions in parallel. Surfaces that may be used in the methods described herein include, but are not limited to, glass or plastic surfaces. Preferably, the surface is a plastic surface, and, more preferably, is one of a plurality of surfaces on a plastic, multi-well plate, such as, for example, a 48-well plate, 96-well plate, 384-well plate, or 1536-well plate, or the like. Even more preferably, the surface is one that has a low amount of auto-fluorescence. Examples of such surfaces include, but are not limited to, white plates, or, preferably, black plastic (e.g., polystyrene) plates, such as OptiPlate 96-well plates (Perkin-Elmer, Boston, Mass.) or black plates by Pierce Biotechnology (Rockford, Ill.). The plates may be custom-coated. The surface is preferably coated with protein A or protein G.

The surface may be prepared in order to capture a sufficient amount of p27 to be used in the ubiquitination assay. A preferred means of capturing p27 to the surface is by the use of an anti-p27 antibody. Such an antibody may be obtained from commercial sources, such as StressGen (Victoria, British Columbia, Canada), Zymed Laboratories (South San Francisco, Calif.) or BD Biosciences Pharmingen (San Diego, Calif.). Examples of anti-p27 antibodies include, but are not limited to, antibodies designated sc-528, sc-527, and K25020.

Alternatively, such an antibody may be prepared as needed using well-known techniques. To produce p27 antibodies, the whole p27 protein may be used; however, a fragment of the protein may also be used. In one embodiment, the antibody is produced through the use of the C-terminal 19 residues of p27 as an immunogen to generate antibodies which immunospecifically bind such an immunogen. p27 antibodies useful in the invention include but are not limited to polyclonal antibodies, monoclonal antibodies, chimeric antibodies, single chain antibodies, Fab fragments, and an Fab expression library. Preferably, the p27 antibody is a monoclonal or polyclonal antibody. In one embodiment, the antibody is antibody sc-528, a rabbit polyclonal antibody raised from a C-terminal peptide (19-mer) of p27.

Various procedures known in the art may be used for the production of polyclonal antibodies to p27 or a fragment thereof. In a particular embodiment, rabbit polyclonal antibodies can be obtained (Pagano, 1995, “From peptide to purified antibody,” in Cell Cycle: Materials and Methods, Pagano, ed., Spring-Verlag., pp. 217-281).

For the production of an anti-p27 antibody, various host animals can be immunized by injection with the native p27, or a synthetic version, or derivative (e.g., fragment) thereof, including, but not limited to, rabbits, mice, and rats. Various adjuvants may be used to increase the immunological response, depending on the host species, and including but not limited to Freund's (complete and incomplete), mineral gels such as aluminum hydroxide (alum), lipopolysaccharide derivatives, such as monophosphoryl lipid A (MPL), surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward p27, any technique which provides for the production of antibody molecules by continuous cell lines in culture may be used. For example, the hybridoma technique originally developed by Kohler and Milstein (Nature 256:495-497 (1975)), as well as the trioma technique, the human B-cell hybridoma technique (Kozbor et al., Immunology Today 4:72 (1983)), and the EBV-hybridoma technique to produce human monoclonal antibodies (Cole, in Monoclonal Antibodies and Cancer Therapy, Alan R. Liss, Inc., pp. 77-96 (1985)). In an additional embodiment of the invention, monoclonal antibodies can be produced in germ-free animals utilizing recent technology (see, e.g., PCT/US90/02545). Human antibodies may be used and can be obtained by using human hybridomas (Cote et al. Proc. Natl. Acad. Sci. USA. 80:2026-2030 (1983)) or by transforming human B cells with Epstein-Barr virus in vitro (Cole et al., in Monoclonal Antibodies and Cancer Therapy, Alan R. Liss, pp. 77-96 (1985)).

Single-chain antibodies may also be used to capture p27. See U.S. Pat. No. 4,946,778.

In the production of anti-p27 antibodies, screening for the desired antibody can be accomplished by techniques known in the art, e.g. ELISA (enzyme-linked immunosorbent assay).

Binding of the antibody to the surface may be accomplished by any means that enables the bound antibody to subsequently bind (i.e., capture) p27 to the surface. For example, biotin-conjugated antibodies may be bound to an avidin-coated surface. In a preferred embodiment, the surface is coated with Protein A or Protein G, and the antibody is contacted with the coated plate and allowed to bind. The choice of Protein A or Protein G to use as a surface coating may depend upon the source of the anti-p27 antibody. For example, Protein A and Protein G bind rabbit and human IgG₁ antibodies approximately equally well; however, Protein G binds sheep, goat and bovine-derived antibodies better than Protein A. Protein A and Protein G may be obtained from Amersham Biosciences, Inc. (Uppsala, Sweden) or from BioVision (Mountain View, Calif.). Protein-A coated plates may be obtained from, for example, Pierce Biotechnology (Rockford, Ill.).

To attach anti-p27 antibodies to surfaces coated with Protein A or Protein G, generally contacting the antibody to the surface is sufficient for binding. In a specific embodiment, the anti-p27 antibody is bound to a Protein A or Protein G-coated surface as follows. The surface is washed one or more times with a buffer containing a mild detergent (e.g., phosphate-buffered saline containing 0.05% Tween 20), after which a solution containing the antibody is deposited onto the surface and allowed to incubate at room temperature for one or more hours, preferably overnight. Excess (i.e., unbound) antibody is washed from the surface with a buffer containing a blocking agent, for example, an ELISA assay diluent (e.g., from BD Pharmingen, No. 55521.3); the buffer or diluent is contacted with the antibody-bound surface and incubated at room temperature for 1-4 hours.

5.1.2 FRET Assay

The p27 ubiquitination assay described herein may also be performed as a FRET (fluorescence resonance energy transfer) assay, preferably a, HTR-FRET (homogenous time-resolved fluorescence resonance energy transfer) assay. In this embodiment, the assay uses two fluorophores and does not require capture to a plate or washing steps.

The assay generally proceeds in two steps. First, the protein components of the assay (e.g., p27, Cdk2, Cyc E, Cks1, Skp1, Skp2, Cul1, Roc1) are brought together generally as described above for the Plate Capture Assay to form a ubiquitination mixture. Typically, Cul1, Roc 1, Skp1 and Skp2 are brought together to form a tetrameric complex to which the other assay components are added. Alternatively, Cul1/Roc1 and Skp1/Skp2 may be brought together to form a dimeric complex, to which the remaining assay components are added. p27 is phosphorylated to make it a suitable acceptor of ubiquitins. Typically, Cks1 and ubiquitin are added last to the ubiquitination mixture. E1 and E2 together, under the appropriate conditions, ubiquitinate p27. Second, after ubiquitination, a component of the p27 ubiquitination complex, preferably p27, is labeled with a first detectable label, and at least one of the ubiquitins attached to p27 is labeled either prior to ubiquitination, or subsequent to ubiquitination, with a second label. The labels are then detected. The extent of labeling is determined by, e.g., detecting the degree of color if the dye is colorimetric, the degree of fluorescence if the label is a fluorophore, etc.). The higher the ratio of the level or amount of p27 labeling to the level or amount of ubiquitin labeling, the greater the amount or degree of ubiquitination of p27. A specific example of the HTR-FRET assay of the invention is described in Example 4.

In one embodiment, both the first label and second label are fluorophores. In a more specific embodiment, said fluorophores are suitable for use in a fluorescence resonance transfer assay. Preferably, one of the first and second labels is a donor fluorophore, and the other label is an acceptor fluorophore. Preferably, the acceptor fluorophore becomes detectable, or becomes substantially more detectable, when proximal to the donor fluorophore (that is, when the donor fluorophore and acceptor fluorophore are present in the same p27 ubiquitination complex, or present on the same ubiquitinated p27). Preferably, the second label is not detectable unless proximal to the first label. Preferably, the acceptor fluorophore is not detectable until the donor fluorophore is excited. The amount of ubiquitinated p27, or the degree or amount of ubiquitination of p27, is determined by determining the ratio of an amount of the acceptor to the amount of the donor label, or of a signal from the acceptor to a signal from the donor label, wherein a higher ratio indicates a greater degree or amount of ubiquitination of p27.

In a more specific embodiment, said p27 is labeled with a first label (e.g., a fluorophore), and said ubiquitin is labeled with a second label (e.g., fluorophore). In a more specific embodiment, said first fluorophore is an acceptor fluorophore, and said second fluorophore a donor fluorophore. In another more specific embodiment, said first fluorophore is a donor fluorophore, and said second fluorophore an acceptor fluorophore. In a more specific embodiment, said acceptor fluorophore fluoresces only when said donor fluorophore is excited. In a more specific embodiment, the first label is europium and the second label is Cy5. In another more specific embodiment, the second label is europium and the first label is Cy5. Eu concentrations should be in the low nM range (e.g., less than 50 nM) while Cy5 concentrations can be up to 100-200 nM. In another more specific embodiment, said donor fluorophore is europium and said acceptor fluorophore is Cy5. In a more specific embodiment, the first label (e.g., Europium) is excited by radiation at 340 nm, the fluorescence of the first label at 620

nm is determined to produce a first fluorescence value, the fluorescence of the second label at 665 nm is determined to produce a second fluorescence value, and the ratio of the second fluorescence value to the first fluorescence value is determined, wherein a higher ratio indicates a greater degree or amount of p27 ubiquitination.

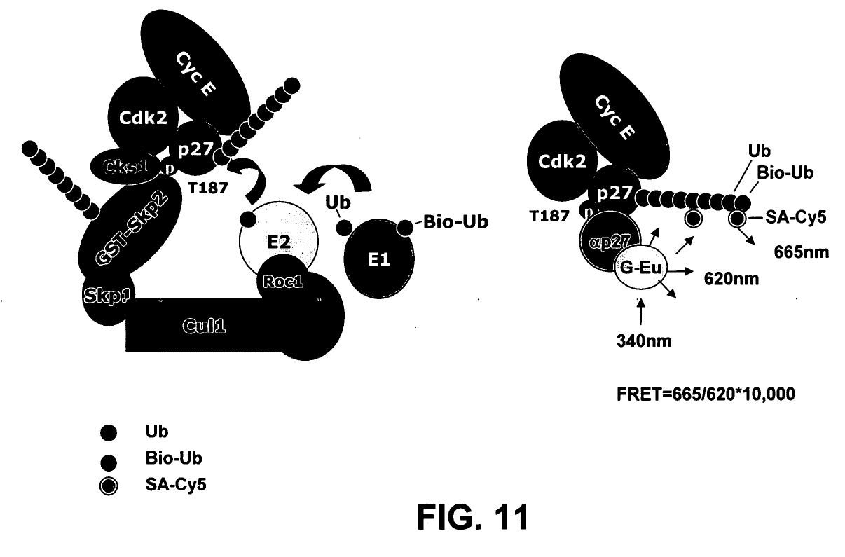

Labeling of the p27 may be accomplished by any art-known method for labeling proteins. A preferred method is to use a labeled anti-p27 antibody or antibody fragment, as described above (see Section 5.1.1.2). While any label may be used, preferably the label is one suitable for use in a fluorescence resonance energy transfer assay. In a preferred embodiment, the p27 is labeled using an anti-p27 antibody to which is coupled a europium-labeled protein G (see, e.g., FIG. 11). The anti-p27 antibody itself may be Eu-labeled. The p27 may also be labeled by attaching a tag, such as hemaglutinin (HA), Myc, Flag, glutathione-S-transferase (GST), and the like, to p27, and binding a labeled antibody to the tag. The p27 may also be expressed as a tagged protein (e.g., a fusion protein that comprises p27 and the tag), and the fusion protein is then bound by the anti-tag antibody or antibody fragment, which is directly or indirectly labeled. The p27, or tagged p27, may also be labeled through the use of a labeled secondary antibody (e.g., a primary human anti-p27 antibody is labeled by binding of an Eu-labeled anti-human rat, goat, mouse, etc. antibody). The Europium may be substituted with any other time-resolve fluorophore, e.g., Cy5, cryptate (e.g., trisbipyridine europium cryptate), Dylight™ 547 or Dylight™ 647 (Pierce Biotechnology, Rockford, Ill.), allophycocyanine (XL665), and the like.

Labeling of ubiquitin may also be accomplished by any art-known method for labeling proteins. Preferably, the ubiquitin is labeled with a fluorophore suitable for time-resolved FRET, such as Cy5, cryptate (e.g., trisbipyridine europium cryptate), Dylight™ 547 or Dylight™ 647 (Pierce Biotechnology, Rockford, Ill.), allophycocyanine (XL665), and the like. As for p27, labeling of ubiquitin may be direct (e.g., the label is bound covalently or non-covalently directly to the ubiquitin, or to a tag where the ubiquitin is expressed as a tagged fusion protein), or the label may be bound via an intermediary (e.g., labeled anti-ubiquitin antibody, labeled streptavidin bound to biotin-bound ubiquitin, etc.).

5.1.3 Assay Components—Polypeptides

The protein components of the p27 ubiquitin assays described herein (e.g., Sections 5.1.1, 5.1.2) may be individually produced, for example, by recombinant DNA methods, or purified from cell extracts. The protein components of the assay (e.g., E1, E2, etc.), or nucleic acids encoding them, may be from any species from which proteins may be derived. For example, each of the protein components of the assay may be derived from a mammal, a fish, a bird, an invertebrate, etc. Preferred protein components of the assay are those derived from the same species as that of the intended target of the compounds; for example, human protein assay components are preferred where identified compounds would be used to treat humans. However, compounds may be identified to treat a particular species, where one or more of the assay protein components are derived from a different species. In one embodiment, each of the protein components of the assay is a mammalian protein (i.e., is derived from a mammal). In another embodiment, each of the protein components of the assay is a primate protein. In another embodiment, each of the protein components of the assay is a human protein. In another embodiment, the protein components of the assay are individually selected without regard to species of origin. In one embodiment, each of the nucleic acids used to produce a protein component of the assay is a mammalian nucleic acid. In another embodiment, each of the nucleic acids used to produce a protein component of the assay is a primate nucleic acid. In another embodiment, each of the nucleic acids used to produce a protein component of the assay is a human nucleic acid. In another embodiment, the nucleic acids encoding the protein components of the assay are individually selected without regard to species of origin.

In one embodiment of the plate capture format of the assay, phosphorylated p27 is present in the assay at a concentration of about 4 ng/μL; said E1 is present at a concentration of about 5 ng/μL; said E2 is present at a concentration of about 150 ng/μL; said E3 is present at a concentration of about 5 ng/μL; said Cks 1 is present at a concentration of about 0.25 ng/μL; and/or said ubiquitin is present at a concentration of about 250 ng/μL. In one embodiment of the embodiment of the FRET format of the assay, said phosphorylated p27 is present the assay at a concentration of about 4 ng/μL; said E1 is present at a concentration of about 5 ng/μL; said E2 is present at a concentration of about 150 ng/μL; said E3 is present at a concentration of about 12.5 ng/μL; said Cks 1 is present at a concentration of about 0.625 ng/μL; or said ubiquitin is present at a concentration of about 34 ng/μL. In another specific embodiment, said amount of ubiquitinated p27 formed in the presence of said compound is lower the amount of ubiquitinated p27 formed in the absence of said compound. In another specific embodiment, said E1, E2, E3 or Cks1 are recombinantly produced. In another specific embodiment, said E1, E2, E3, and Cks1 are purified from a cell extract. In another specific embodiment, said ubiquitin is labeled. In a more specific embodiment, said label is biotin.

In various embodiments, E1 proteins useful in the invention include, but are not limited to, those having the amino acid sequence of the polypeptide having ATCC accession numbers A38564, S23770, AAA61246, P22314, CAA40296 or BAA33144, which are incorporated herein by reference. E1 is commercially available (e.g., Affiniti Research Products (Exeter, U.K.)).

In various embodiments, nucleic acids that may be used to produce E1 proteins for the invention include, but are not limited to, those disclosed by ATCC accession numbers M58028, X56976 or AB012190, which are incorporated herein by reference.

In various embodiments, E2 proteins that may be used in the present invention include, but are not limited to, those having the amino acid sequences disclosed in ATCC accession numbers AAC37534, P49427, CAA82525, AAA58466, AAC41750, P51669, AAA91460, AAA91461, CAA63538, AAC50633, P27924, AAB36017, Q16763, AAB86433, AAC26141, CAA04156, BAA11675, Q16781 or CAB45853, each of which is incorporated herein by reference.

Nucleic acids that may be used to make E2 include, but are not limited to, those nucleic acids having sequences disclosed in ATCC accession numbers L2205, Z29328, M92670, L40146, U39317, U39318, X92962, U58522, S81003, AF031141, AF075599, AJ000519, or D83004, each of which is incorporated herein by reference.

In a preferred embodiment, E2 has a tag, as defined above. Examples of E2 tags include, but are not limited to, labels, partners of binding pairs and substrate binding elements. In one embodiment, the tag is a His-tag or GST-tag.

The present invention provides methods and compositions comprising E3. By “E3,” it is meant a ubiquitin ligase, as defined above. In one embodiment, E3 comprises a ring finger protein and a Cullin. In various embodiments, ring finger proteins include, but are not limited to, Roc1, Roc2 or APC11.

Other examples of ring finger proteins include, but are not limited to, proteins having the amino acid sequence disclosed in ATCC accession numbers AAD30147 and AAD30146 or 6320196, incorporated herein by reference. Nucleic acids that may be used to make the ring finger proteins include, but are not limited to, those having the nucleic acid sequences disclosed in ATCC accession numbers AF142059, AF142060 or nucleic acids 433493 to 433990 of NC_{—}001136.

In various embodiments, Cullins include, but are not limited to, Cul 1, Cul 2, Cul 3, Cul 4A, Cul 4B, Cul5 or APC2. In one embodiment, the ring finger protein is Roc1 and the Cullin is Cul1. Other examples of Cullins include, but are not limited to, proteins having the amino acid sequences disclosed in ATCC accession numbers 4503161, AAC50544, AAC36681, 4503163, AAC51190, AAD23581, 4503165, AAC36304, AAC36682, AAD45191, AAC50548, Q13620, 4503167 or AAF05751, each of which is incorporated herein by reference.

Any one of the proteins used in the assay can comprise a tag, for example, a short polypeptide sequence facilitating or enabling isolation or purification.

Expression and purification of the recombinant or wild-type polypeptides can be achieved by any conventional methods known in the art (e.g., Sambrook et al., Molecular Cloning, A Laboratory Manual, 3^{rd} Ed. (2001); Kriegler, Gene Transfer and Expression, A Laboratory Manual (1990); Current Protocols in Molecular Biology (1994); Scopes, Protein Purification: Principles and Practice, 3^{rd} Ed. (1994); and Deutscher, Guide to Protein Purification (1990)), as well as those described herein. The compounds of the present invention can be extracted and purified from the culture media or a cell by using known protein purification techniques commonly employed, such as extraction, precipitation, ion exchange chromatography, affinity chromatography, gel filtration and the like. Compounds can be isolated by affinity chromatography using the modified receptor protein extracellular domain bound to a column matrix or by heparin chromatography.

Any of the polypeptides that are used in the high-throughput assay described herein can include conservative variations of the native polypeptide sequence. The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

Minor modifications of the primary amino acid sequences of any of the above polypeptide may result in a polypeptide that has substantially equivalent activity as compared to the unmodified counterpart polypeptide described herein. Such modifications may be deliberate, as by site-directed mutagenesis, or may be spontaneous. Any modified version of the above-listed polypeptides produced by these modifications may be used in the assay described herein as long as the biological activity of the polypeptide still exists.

Deletion of one or more amino acids can also result in a modification of the structure of the resultant molecule without significantly altering its activity. Deletion can lead to the development of a smaller active molecule-which could have broader utility. For example, it may be possible to remove amino or carboxy terminal amino acids and retain native activity, or an activity substantially similar to that of the native protein.

In addition to sequence variants, fragments or derivatives of any of the protein components of the assay may be used, as long as a substantial portion of the native protein's activity is retained. For example, fragments or variants of any or all of the components of the substrate (p27/Cdk2/Cyclin E, as described below) may be used in place of the native protein(s), as long as the combination of proteins and fragments, or combination of fragments, acts as a substrate for an E3 ligase, and the p27 or p27 fragment is competent to be ubiquitinated under conditions in which native p27, in conjunction with native Cdk2 and native Cyclin E, would be ubiquitinated.

5.1.4 Visualization

According to the method of the present invention, the extent of ubiquitination of p27 is determined by labeling ubiquitin such that the ubiquitin is detectable by standard means. As used herein, and unless otherwise specified, the term “label” means a composition or compound detectable by, e.g., spectroscopic, photochemical, biochemical, immunochemical, or other chemical means.

In one embodiment, the ubiquitin is visualized using a fluorophore as a fluorescent label. By “fluorescent label” is meant any molecule that may be detected via its inherent fluorescent properties. In a specific embodiment, said fluorophore is Europium (Eu). See Hemmila, “Europium as a label in time-resolved immunofluorometric assays,” Anal Biochem., 137(2):335-43 (1984). Other fluorophores may be used as fluorescent labels. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™ and Texas Red. Suitable optical dyes are described in, for example, Haugland, Molecular Probes Handbook (1996), hereby expressly incorporated by reference. Other suitable fluorescent labels include, but are not limited to, green fluorescent protein (GFP: Chalfie, et al., Science, 263(5148): 802-805 (1994); and EGFP; Clontech—Genbank Accession Number U55762), blue fluorescent protein (BFP: Quantum Biotechnologies, Inc., 1801 de Maisonneuve Blvd. West, 8th Floor, Montreal (Quebec) Canada H3H 1J9; Stauber, R. H., Biotechniques, 24(3): 462-471 (1998); Heim, R. and Tsien, R. Y., Curr. Biol., 6: 178-182 (1996)), enhanced yellow fluorescent protein (EYFP: Clontech Laboratories, Inc., 1020 East Meadow Circle, Palo Alto, Calif. 94303), luciferase (Ichiki et al., J. Immunol., 150(12): 5408-5417 (1993)), β-galactosidase (Nolan et al., Proc Natl Acad Sci USA, 85(8): 2603-2607 (1988); WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558). All of the above-cited references are expressly incorporated herein by reference.

In another embodiment, the label used to visualize ubiquitin is a fluorophore suitable for use in fluorescence resonance energy transfer. The fluorophore used to label ubiquitin may be an acceptor fluorophore or a donor fluorophore; in a preferred embodiment, another protein in the assay, e.g., p27, is labeled with a FRET-suitable fluorophore as well. Where ubiquitin in ubiquitinated p27 is labeled with a donor or acceptor fluorophore, and p27 is labeled with an acceptor or donor fluorophore, respectively, excitation of the donor causes fluorescence of the acceptor fluorophore. The ubiquitin may be labeled with a FRET-compatible fluorophore that causes, or reacts, by extinction rather than fluorescence.

Other suitable labels include, but are not limited to, radioisotopes (e.g., ³H, ³²P, ³C, ¹¹C, ¹⁴C, ³⁵S), magnetic beads (e.g., DYNABEADS™), enzymes (e.g., horse radish peroxidase, alkaline phosphatase, and others commonly used in an ELISA), calorimetric labels (e.g., colloidal gold, colored glass, plastic beads such as, but not limited to, polystyrene, polypropylene, and latex), electron dense reagents, biotin, digoxigenin, haptens and proteins for which anti-sera or monoclonal antibodies are available, and other labels that can be detected by mass spectroscopy, NMR spectroscopy, or other well-known analytical methods. In one embodiment, ubiquitin is visualized using biotin as a label.

5.1.5 Screening Test Compounds

The p27 ubiquitination assays described above can be used to screen compounds for their ability to modulate the ubiquitination of p27. Such compounds would potentially be highly useful in the treatment of cancers, proliferative disorders, and other diseases disorders or conditions associated with the ubiquitin-mediated degradation of p27, with the concomitant loss of control of the cell cycle and development of, e.g., inappropriate proliferation or metastasis. Such compounds may, for example, be contacted with p27 after phosphorylation and before contacting with the ubiquitinating components of the assay, or before phosphorylation. The assay may therefore be used to identify compounds that modulate the phosphorylation of p27, thus modulating the rate of formation of the ubiquitinizable precursor to p27-Ub, or may be used to identify compounds that modulate the rate of ubiquitination of phosphorylated p27, thus modulating the formation of p27-Ub.

The invention provides a method of identifying a compound that modulates ubiquitination of p27, comprising ubiquitinating p27 in the presence and in the absence of the compound, and detecting a difference in the degree or amount of ubiquitination of p27 in the presence of the compound compared to the degree or amount of ubiquitination of p27 in the absence of the compound. In one embodiment, the invention provides a method of identifying a compound that modulates ubiquitination of p27 comprising ubiquitinating p27 in the presence of the compound to produce a first p27 sample; ubiquitinating p27 in the absence of the compound to produce a second p27 sample, and determining an amount of ubiquitination of said first p27 sample and of said second p27 sample, wherein the compound modulates ubiquitination if the amounts of ubiquitination obtained from the first and second samples are different.

In one embodiment using a plate capture, the invention provides a method of identifying a compound that modulates ubiquitination of p27 comprising preparing a first sample by combining an amount of p27 with a candidate compound to form a mixture of p27 and the compound; preparing a second sample comprising an amount of p27 in the absence of the compound; respectively contacting the first and second samples with a plurality of polypeptides, said plurality of peptides together capable of ubiquitinating p27, to form ubiquitinated p27; respectively capturing the ubiquitinated p27 in the first and second samples on a surface; and respectively determining the amounts of ubiquitin present in p27 in the first and second samples, captured on the surface, wherein the compound modulates ubiquitination if the amounts of ubiquitination obtained from the first and second samples are different.

In another embodiment using FRET, the invention provides a method of identifying a compound that modulates ubiquitination of p27 comprising ubiquitinating p27 with ubiquitin in the presence of the compound to produce a first ubiquitinated p27; ubiquitinating p27 in the absence of the compound to produce a second ubiquitinated p27, labeling said p27 in said first and second ubiquitinated p27 with a first label, labeling said ubiquitin in said first and second ubiquitinated p27 with a second label; determining for both said first ubiquitinated p27 and said second ubiquitinated p27 a ratio of said first label to said second label to produce a first ratio and a second ratio, respectively; wherein the compound modulates ubiquitination if said first ratio and said second ratio are different.

In a specific embodiment, said p27 that is contacted with said compound is phosphorylated p27. In another specific embodiment, said p27 that is contacted with said compound is unphosphorylated p27. In another specific embodiment, said compound is contacted with p27, E1, E2, E3, Cks1, ubiquitin, or any combination thereof. In another specific embodiment, said compound is a polypeptide, a polynucleotide, a polysaccharide, a lipid, or a combination thereof. In another specific embodiment, said compound is a small organic molecule or a drug. In another specific embodiment, said compound has a known activity different than modulating the level or amount of ubiquitination of p27. In another specific embodiment, said compound has no known activity other than modulation of ubiquitination of p27.

In a specific embodiment of either of the plate or FRET assays above, said first sample and said second sample are prepared by mixing an amount of p27, E1, E2, E3, Cks1, ubiquitin or any mixture thereof, with, in the case of the first sample, a candidate agent. In a specific embodiment, the method can be used to identify a prospective anti-cancer agent, where the compound decreases ubiquitination of p27 relative to the condition in which the compound is not present. In another specific embodiment, the method can be used to identify a prospective anti-proliferative agent, where the compound decreases ubiquitination of p27 relative to the condition in which the compound is not present. In another specific embodiment, the method can be used to identify a prospective compound effective against a disease, condition or disorder, where the disease, condition or disorder is associated with an increased ubiquitination of p27 as compared to a normal cell.

In another embodiment, the invention provides a method of identifying an anticancer agent, comprising determining the amount of ubiquitinated p27 formed by combining isolated p27, E1, E2, E3, Cks1, Cyclin E, Cdk2 and ubiquitin in the presence of said compound and in the absence of said compound; wherein, if the amount of ubiquitinated p27 formed in the presence of said compound differs from the amount of ubiquitinated p27 formed in the absence of said compound, said compound is identified as an anticancer agent. In a specific embodiment, said p27 is phosphorylated with Cdk2 and Cyclin E prior to combination with said E1, E2, E3, Cdk2 and ubiquitin.

In another specific embodiment, said phosphorylated p27 is present at a concentration of about 4 ng/μL; said E1 is present at a concentration of about 5 ng/μL; said E2 is present at a concentration of about 150 ng/μL; said E3 is present at a concentration of about 5 ng/μL; said Cks2 is present at a concentration of about 0.25 ng/μL; and/or said ubiquitin is present at a concentration of about 500 ng/μL. In another specific embodiment, said phosphorylated p27 is present at a concentration of about 4 ng/μL; said E1 is present at a concentration of about 5 ng/μL; said E2 is present at a concentration of about 150 ng/μL; said E3 is present at a concentration of about 12.5 ng/μL; said Cks 1 is present at a concentration of about 0.625 ng/μL; or said ubiquitin is present at a concentration of about 34 ng/μL. In another specific embodiment, said amount of ubiquitinated p27 formed in the presence of said compound is lower the amount of ubiquitinated p27 formed in the absence of said compound. In another specific embodiment, said E1, E2, E3 or Cks1 are recombinantly produced. In another specific embodiment, said E1, E2, E3, and Cks1 are purified from a cell extract. In another specific embodiment, said ubiquitin is labeled. In a more specific embodiment, said label is biotin.

Because the assay uses individual components that together ubiquitinate p27, and because the assay, either the plate capture assay or FRET assay, are suitable for multiple simultaneous performance, the assay methods disclosed herein achieve consistent, reproducible results and are uniquely suitable for high-throughput uses, e.g., screening chemical libraries to identify modulators of p27 ubiquitination.

Preferably, the compound(s) to be tested for p27 ubiquitination modulation activity are small, organic molecules. Such, molecules may be readily identified, for example, using libraries of compounds (e.g., combinatorial libraries). As used herein, the term “library” refers to a plurality of compounds, and “combinatorial library” means, e.g., a collection of compounds synthesized using combinatorial chemistry techniques, or a collection of unique chemicals of low molecular weight (less than 1000 Daltons) that each occupy a unique three-dimensional space. In specific embodiments, a library comprises 50; 100; 150; 200; 250; 500; 750; 1,000; 1,250; 1,500; 1,750; 2,000; 2,500; 5,000; 7,500; 10,000; 20,000; 30,000; 40,000; or 50,000 different compounds. In other specific embodiments, a library comprises at most 50; 100; 150; 200; 250; 500; 750; 1,000; 1,250; 1,500; 1,750; 2,000; 2,500; 5,000; 7,500; 10,000; 20,000; 30,000; 40,000; or 50,000 different compounds. In other specific embodiments, a library comprises between 10 and 100; 10 and 150; 100 and 200; 100 and 250; 100 and 500; 100 and 750; 500 and 1,000; 500 and 1,250; 500 and 1,500; 500 and 1,750; 1,000 and 2,000; 1,000 and 2,500; 2,000 and 5,000; 2,000 and 7,500; 2,000 and 10,000; 5,000 and 20,000; 10,000 and 30,000; 10,000 and 40,000; 20,000 and 50,000; 10,000 and 100,000; 20,000 and 200,000; 30,000 and 300,000; 40,000 and 400,000; or between 50,000 and 500,000 different compounds. It has been observed that the systematic, combinatorial mixing of 100 interchangeable chemical building blocks results in the theoretical synthesis of 100 million tetrameric compounds or 10 billion pentameric compounds. (Gallop et al., “Applications of Combinatorial Technologies to Drug Discovery, Background and Peptide Combinatorial Libraries,” J. Med. Chem., 37(9):1233-1250 (1994)).

Other chemical libraries known to those in the art may also be used, for example, natural product libraries. For example, a linear combinatorial chemical library such as a polypeptide library may formed by combining amino acids in every possible combination to yield peptides of a given length; the peptides are then tested for p27 ubiquitination modification activity.

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 pooled compounds that can be screened by administration to a single reaction well. Thus, if modulation is detected, smaller and smaller pools of interacting pairs can be assayed for the modulation activity. By such methods, many candidate molecules can be screened.

Methods of constructing libraries of compounds suitable for testing in the p27 ubiquitination assay described herein are known in the art. For example, examples of peptide libraries are given in Houghten et al., Nature, 354: 84-86 (1991), which describes mixtures of free hexapeptides in which the first and second residues in each peptide were individually and specifically defined; Lam et al., Nature, 354: 82-84 (1991), which describes a “one bead, one peptide” approach in which a solid phase split synthesis scheme produced a library of peptides in which each bead in the collection had immobilized thereon a single, random sequence of amino acid residues; Medynski, Bio/Technology, 12: 709-710 (1994), which describes split synthesis and T-bag synthesis methods; and Gallop et al., J. Medicinal Chemistry, 37(9): 1233-1251 (1994). Combinatorial libraries may also be prepared according to the methods of Ohlmeyer et al., Proc. Natl. Acad. Sci. USA, 90: 10922-10926 (1993); Erb et al., Proc. Natl. Acad. Sci. USA, 91: 11422-11426 (1994); Houghten et al., Biotechniques, 13: 412 (1992); Jayawickreme et al., Proc. Natl. Acad. Sci. USA, 91: 1614-1618 (1994); or Salmon et al., Proc. Natl. Acad. Sci. USA, 90: 11708-11712 (1993). PCT Publication No. WO 93/20242, and Brenner and Lerner, Proc. Natl. Acad. Sci. USA, 89: 5381-5383 (1992) describe “encoded combinatorial chemical libraries,” that contain oligonucleotide identifiers for each chemical polymer library member.

Libraries of non-peptides, e.g., peptide. derivatives (for example, that contain one or more non-naturally occurring amino acids) can also be used. See, e.g., Simon et al., Proc. Natl. Acad. Sci. USA, 89: 9367-9371 (1992).

Multiple combinatorial chemistry libraries may be used to identify particularly useful compounds having p27 modulation activity. For example, a library of small molecules may be generated using methods of combinatorial library formation well known in the art. See e.g., U.S. Pat. Nos. 5,463,564 and 5,574, 656, the disclosures of which are incorporated herein by reference in their entireties. Library compounds are screened using the assay described herein to identify those compounds that possess desired structural and functional properties. The characteristics of each library compound are encoded so that compounds demonstrating ubiquitination modulation activity can be analyzed and features common to the various compounds identified can be isolated and combined into future iterations of libraries.

Once a library of compounds is screened, subsequent libraries may be generated using those chemical building blocks that possess the features shown in the first round of screen to modulate the ubiquitination of p27. Using this method, subsequent iterations of candidate compounds will possess more and more of those structural and functional features required to modulate the ubiquitination of p27, until a group of test compounds with a high degree of activity can be found. These compounds can then be further tested for their safety and efficacy as anti-tumor and/or anti-cancer drugs for use in mammals.

As contemplated by the present invention, the assay may be used to identify any compound that modulates, i.e., detectably changes, the ubiquitination, or ubiquitination state, of p27. Modulation of p27 ubiquitination may mean either increasing or decreasing ubiquitination. Preferably, in one embodiment, the identified compound detectably inhibits ubiquitination as compared to a control sample not contacted with the compound. In a specific embodiment, the compound inhibits ubiquitination of p27 by 50% or more. A compound stimulates ubiquitination if the fraction of the substrate that is ubiquitinated or the amount of ubiquitin incorporated into substrate is increased as compared to reactions performed in the absence of the test compound. In one embodiment, the compound stimulates ubiquitination by 50% or more as compared to a control sample not contacted with the compound.

5.1.6 High-Throughput Modes

Assays of the invention, in part because they are performed on a plate platform, are particularly useful for high-throughput screening of compounds. Assays of the invention may employ an array of samples (e.g., ubiquitination reaction mixtures) that comprises, e.g., 24, 36, 48, 96, 300, 500, or 1000 or more samples. Where an array contains a high number of samples, the array may comprise one or more sub-arrays, sub-arrays containing samples comprising different constituents.

Preferably the samples are prepared, added to sample wells and mixed automatically. Similarly, assays can be performed and processed automatically in each samples. As used herein, the term “automated” or “automatically” refers to the use of computer software and/or robotics to add, mix and analyze the samples, components, and specimens.

Samples are added to the sample wells using various deposition or material transfer techniques known in the art, including, but not limited to, hand placement, pipetting, and other manual or automated solid or liquid distribution systems.

After adding and mixing the components to the sample wells, the samples may be processed according to assay methods of the invention. The samples can be processed individually or as a group. A number of microarray systems that can be adapted for use in assay methods of the invention are commercially available. Examples of microarray systems suitable for assay methods of the invention include, but are not limited to, those manufactured by Gene Logic of Gaithersburg, Md. (see U.S. Pat. No. 5,843,767), Luminex Corp., Austin, Tex., Beckman Instruments, Fullerton, Calif., MicroFab Technologies, Plano, Tex., Nanogen, San Diego, Calif., and Hyseq, Sunnyvale, Calif. These devices test samples based on a variety of different systems. All include thousands of microscopic channels that direct components into test wells, where reactions can occur. These systems are connected to computers for analysis of the data using appropriate software and data sets. The Beckman Instruments system can deliver nanoliter samples of 96 or 384-arrays, and MicroFab Technologies system delivers sample using inkjet printers to aliquot discrete samples into wells.

These and other systems can be adapted as required for use herein. For example, the combinations of assay components and test compounds at various concentrations and combinations can be generated using standard formulating software (e.g., Mathlab software, commercially available from Mathworks, Natick, Mass.). The combinations thus generated can be downloaded into a spread sheet, such as Microsoft EXCEL. From the spread sheet, a work list can be generated for instructing the automated distribution mechanism to prepare an array of samples according to the various combinations generated by the formulating software. The work list can be generated using standard programming methods according to the automated distribution mechanism that is being used. The use of work lists simply allows a file to be used as the process command rather than discrete programmed steps. The work list combines the formulation output of the formulating program with the appropriate commands in a file format directly readable by the automatic distribution mechanism.

The automated distribution mechanism delivers at least one component, as well as various additional components, to each sample well. Preferably, the automated distribution mechanism can deliver multiple amounts of each component. In one embodiment, the automated distribution mechanism utilizes one or more micro-solenoid valves.

Automated liquid and solid distribution systems are well known in the art, and commercially available, such as the Tecan Genesis, from Tecan-US, Research Triangle Park, N.C. The robotic arm can collect and dispense individual components, or mixtures thereof, from a stock plate to a sample well or site. The process is repeated until an array is completed. The samples are then mixed. For example, the robotic arm moves up and down in each well plate for a set number of times to ensure proper mixing.

Those of ordinary skill in the art would appreciate that variations of automated high-throughput assays can be made without departing from the scope and spirit of this invention.

5.1.7 Application of Assay to Other Proteins

The assay methods described above may be generalized to assays of the ubiquitination of any protein. For example, a generalized plate capture assay comprises the plates, as described above, comprising a capture antibody specific to the protein of interest; ubiquitination proteins (e.g., a ubiquitin-activating protein, a ubiquitin conjugating protein, and a ubiquitin ligase), and any proteins needed to prepare the protein for ubiquitination. For example, the described method can easily be adapted for detection of IKB ubiquitination and used for screening of compounds that inhibit IKB ubiquitination. In the same manner, the HTR-FRET-format assay may be conducted using the protein of interest, ubiquitination proteins (e.g., a ubiquitin-activating protein, a ubiquitin conjugating protein, and a ubiquitin ligase), any proteins needed to prepare the protein for ubiquitination. Labeling of the protein of interest would be accomplished in the same manner as labeling of p27 using, e.g., an antibody specific for the protein of interest. Labeling of ubiquitin in the ubiquitinated protein of interest is accomplished as described above for the p27 FRET assay. A determination of the effect of a compound on the ubiquitination of a protein of interest, for either the plate capture or FRET assay, is conducted in the same manner as described herein for p27.

5.2 Applications

As stated above, the method of the present invention is useful for the identification of one or more compounds (e.g., polypeptides, polynucleotides, lipids, polysaccharides, small organic molecules, drugs or drug candidates, etc.) that may be use to treat a cancer that results, in whole or in part, from a change in the ubiquitination of p27. Such cancers include, but are not necessarily limited to, leukemias, including acute leukemia, acute lymphocytic leukemia, acute myelocytic leukemia, myeloblastic leukemia, promyelocytic leukemia, myelomonocytic leukemia, monocytic leukemia, and erythroleukemia; chronic leukemia, such as chronic myelocytic (granulocytic) leukemia or chronic lymphocytic leukemia; polycythemia vera; lymphomas, such as Hodgkin's disease and non-Hodgkin's disease; multiple myeloma; Waldenstrom's macroglobulinemia; heavy chain disease; solid tumors, such as sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, or retinoblastoma; etc.

The assay is also useful for identifying compounds that are useful for the treatment of other disorders or conditions, whether cancer-related or not, related to changes in ubiquitination of p27. In various embodiments, such disorders or conditions include, but are not limited to, proliferative disorders and dysplasias, T cell and fibroblast proliferation and proliferation-related disorders; endometriosis, inflammation, etc.

Once a compound, or set of compounds, able to modulate the ubiquitination of p27, preferably able to reduce the ubiquitination of p27, has been identified, such compounds may be further tested in vitro on the one or more cell types related to, or responsible for, the particular cancer, disease, condition or disorder related to p27 ubiquitination. For example, a compound identified as a p27 ubiquitination modulator may be tested in vitro to determine the effect on metastatic cancer cells or inappropriately-proliferating cells to determine if there is any consequent change in the metastatic or proliferative behavior of the cells. A positive or desirable change is the lessening or elimination of metastasis or inappropriate proliferation.

It is preferable also to perform an assay, such as the assay described herein, to establish the state of p27 ubiquitination in both the compound-receiving and control cell groups. Alternatively, one may perform an antibody-based experiment to determine the effect of the compound(s) on the levels of p27 in the cells. Such antibody-based assays may be performed by methods known in the art (ELISA, RIA, etc.). For such experiments, either p27 levels in equivalent, normal cells, or a standard p27 level, may be used as a control.

A compound identified in the high-throughput assay described herein (an “identified compound”) may also be tested in vivo to determine its effect on a particular cancer, disease, disorder or condition of interest. For example, identified compounds may be tested at varying concentrations using known mouse, or other mammal, tumor models, or may be administered in varying concentrations to a mammal, such as a mouse, rat, rabbit, etc., before, at the same time as, or subsequent to administration of tumor cells for the particular cancer from the particular mammal used as a model.

Where the assay is used to identify potential anti-tumorigenic or antiproliferative compounds, compounds identified by the assay of the present invention may be demonstrated to inhibit tumor cell proliferation, cell transformation and tumorigenesis in vitro or in vivo using a variety of assays known in the art, or described herein. Such assays may use cells of a cancer cell line or cells from a patient. Many assays well-known in the art can be used to assess such survival and/or growth; for example, cell proliferation can be assayed by measuring (³H)-thymidine incorporation, by direct cell count, by detecting changes in transcription, translation or activity of known genes such as proto-oncogenes (e.g., fos, myc) or cell cycle markers (Rb, cdc2, cyclin A, D1, D2, D3 or E). The levels of such protein and mRNA and activity can be determined by any method well known in the art. For example, protein can be quantitated by known immunodiagnostic methods such as western blotting or immunoprecipitation using commercially available antibodies (for example, many cell cycle marker antibodies are from Santa Cruz, Inc.). mRNA can be quantitated by methods that are well known and routine in the art, for example by northern analysis, RNase protection, the polymerase chain reaction in connection with the reverse transcription, etc. Cell viability can be assessed by using trypan-blue staining or other cell death or viability markers known in the art. Differentiation can be assessed visually based on changes in morphology, etc.

Cell cycle and cell proliferation analysis may be performed by a variety of techniques known in the art, including but not limited to the following:

As one example, bromodeoxyuridine (“BRDU”) incorporation may be used as an assay to identify proliferating cells. The BRDU assay identifies a cell population undergoing DNA synthesis by incorporation of BRDU into newly synthesized DNA. Newly synthesized DNA may then be detected using an anti-BRDU antibody (see Hoshino et al., 1986, Int. J. Cancer 38, 369; Campana et al., 1988, J. Immunol. Meth. 107, 79).

Cell proliferation may also be examined using (3H)-thymidine incorporation (see, e.g., Chen, 1996, Oncogene 13:1395-403; Jeoung, 1995, J. Biol. Chem. 270:18367-73). This assay allows for quantitative characterization of S-phase DNA synthesis. In this assay, cells synthesizing DNA will incorporate (3H)-thymidine into newly synthesized DNA. Incorporation may then be measured by standard techniques in the art such as by counting of radioisotope in a Scintillation counter (e.g., Beckman LS 3800 Liquid Scintillation Counter).

Detection of proliferating cell nuclear antigen (PCNA) may also be used to measure cell proliferation. PCNA is a 36 kilodalton protein whose expression is elevated in proliferating cells, particularly in early G1 and S phases of the cell cycle and therefore may serve as a marker for proliferating cells. Positive cells are identified by immunostaining using an anti-PCNA antibody (see Li et al., 1996, Curr. Biol. 6:189-199; Vassilev et al., 1995, J. Cell Sci. 108:1205-15).

Cell proliferation may be measured by counting samples of a cell population over time (e.g., daily cell counts). Cells may be counted using a hemacytometer and light microscopy (e.g., HyLite hemacytometer, Hausser Scientific). Cell number may be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells may be measured, for example, based on the DNA ploidy value of the cell. For example, cells in the G1 phase of the cell cycle generally contain a 2N DNA ploidy value. Cells in which DNA has been replicated but have not progressed through mitosis (e.g., cells in S-phase) will exhibit a ploidy value higher than 2N and up to 4N DNA content. Ploidy value and cell-cycle kinetics may be further measured using propidum iodide assay (see, e.g., Turner, et al., 1998, Prostate 34:175-81). Alternatively, the DNA ploidy may be determined by quantization of DNA Feulgen staining (which binds to DNA in a stoichiometric manner) on a computerized microdensitometry staining system (see, e.g., Bacus, 1989, Am. J. Pathol. 135:783-92). In an another embodiment, DNA content may be analyzed by preparation of a chromosomal spread (Zabalou, 1994, Hereditas. 120:127-40; Pardue, 1994, Meth. Cell Biol. 44:333-351).

The expression of cell-cycle proteins (e.g., CycA, CycB, CycE, CycD, cdc2, Cdk4/6, Rb, p21 or p27) provide crucial information relating to the proliferative state of a cell or population of cells. For example, identification in an anti-proliferation signaling pathway may be indicated by the induction of p21cip1. Increased levels of p21 expression in cells results in delayed entry into G1 of the cell cycle (Harper et al., 1993, Cell 75:805-816; Li et al., 1996, Curr. Biol. 6:189-199). p21 induction may be identified by immunostaining using a specific anti-p21 antibody available commercially (e.g., from Santa Cruz, Inc.). Similarly, cell-cycle proteins may be examined by Western blot analysis using commercially available antibodies. In another embodiment, cell populations are synchronized prior to detection of a cell cycle protein. Cell-cycle proteins may also be detected by FACS (fluorescence-activated cell sorter) analysis using antibodies against the protein of interest.

Detection of changes in length of the cell cycle or speed of cell cycle may also be used to measure inhibition of cell proliferation by a identified compounds. In one embodiment the length of the cell cycle is determined by the doubling time of a population of cells (e.g., using cells contacted or not contacted with one or more identified compounds). In another embodiment, FACS analysis is used to analyze the phase of cell cycle progression, or purify G1, S, and G2/M fractions (see, e.g., Delia et al., 1997, Oncogene 14:2137-47).

Lapse of cell cycle checkpoint(s), and/or induction of cell cycle checkpoint(s), may be examined by the methods described herein, or by any method known in the art. Without limitation, a cell cycle checkpoint is a mechanism which ensures that a certain cellular events occur in a particular order. Checkpoint genes are defined by mutations that allow late events to occur without prior completion of an early event (Weinert and Hartwell, 1993, Genetics, 134:63-80). Induction or inhibition of cell cycle checkpoint genes may be assayed, for example, by western blot analysis, or by immunostaining, etc. Lapse of cell cycle checkpoints may be further assessed by the progression of a cell through the checkpoint without prior occurrence of specific events (e.g. progression into mitosis without complete replication of the genomic DNA).

In addition to the effects of expression of a particular cell cycle protein, activity and post-translational modifications of proteins involved in the cell cycle can play an integral role in the regulation and proliferative state of a cell. The invention provides for the use of assays involving detection of post-translational modifications (e.g., phosphorylation) by any method known in the art. For example, antibodies that detect phosphorylated tyrosine residues are commercially available, and may be used in western blot analysis to detect proteins with such modifications. In another example, modifications such as myristylation, may be detected on thin layer chromatography or reverse phase H.P.L.C. (see, e.g., Glover, 1988, Biochem. J. 250:485-91).

Activity of signaling and cell cycle proteins and/or protein complexes is often mediated by a kinase activity. The present invention provides for analysis of kinase activity by assays such as the histone H1 assay (see e.g., Delia et al., 1997, Oncogene 14:2137-47).

The identified compounds can also be demonstrated to alter cell proliferation in cultured cells in vitro using methods which are well known in the art. Specific examples of cell culture models include, but are not limited to, for lung cancer, primary rat lung tumor cells (Swafford et al., 1997, Mol. Cell. Biol., 17:1366-1374) and large-cell undifferentiated cancer cell lines (Mabry et al., 1991, Cancer Cells, 3:53-58); colorectal cell lines for colon cancer (Park and Gazdar, 1996, J. Cell Biochem. Suppl. 24:131-141); multiple established cell lines for breast cancer (Hambly et al., 1997, Breast Cancer Res. Treat. 43:247-258; Gierthy et al., 1997, Chemosphere 34:1495-1505; Prasad and Church, 1997, Biochem. Biophys. Res. Commun. 232:14-19); a number of well-characterized cell models for prostate cancer (Webber et al., 1996, Prostate, Part 1, 29:386-394; Part 2, 30:58-64; and Part 3, 30:136-142; Boulikas, 1997, Anticancer Res. 17:1471-1505); for genitourinary cancers, continuous human bladder cancer cell lines (Ribeiro et al., 1997, Int. J. Radiat. Biol. 72:11-20); organ cultures of transitional cell carcinomas (Booth et al., 1997, Lab Invest. 76:843-857) and rat progression models (Vet et al., 1997, Biochim. Biophys Acta 1360:39-44); and established cell lines for leukemias and lymphomas (Drexler, 1994, Leuk. Res. 18:919-927, Tohyama, 1997, Int. J. Hematol. 65:309-317).

Identified compounds can also be demonstrated to inhibit cell transformation (or progression to malignant phenotype) in vitro. In this embodiment, cells with a transformed cell phenotype are contacted with one or more identified compounds, and examined for change in characteristics associated with a transformed phenotype (a set of in vitro characteristics associated with a tumorigenic ability in vivo), for example, but not limited to, colony formation in soft agar, a more rounded cell morphology, looser substratum attachment, loss of contact inhibition, loss of anchorage dependence, release of proteases such as plasminogen activator, increased sugar transport, decreased serum requirement, or expression of fetal antigens, etc. (see Luria et al., 1978, General Virology, 3d Ed., John Wiley & Sons, New York, pp. 436-446).

Loss of invasiveness or decreased adhesion may also be used to demonstrate the anti-cancer effects of the identified compounds. For example, a critical aspect of the formation of a metastatic cancer is the ability of a precancerous or cancerous cell to detach from primary site of disease and establish a novel colony of growth at a secondary site. The ability of a cell to invade peripheral sites is reflective of a potential for a cancerous state. Loss of invasiveness may be measured by a variety of techniques known in the art including, for example, induction of E-cadherin-mediated cell-cell adhesion. Such E-cadherin-mediated adhesion can result in phenotypic reversion and loss of invasiveness (Hordijk et al., 1997, Science 278:1464-66).

Loss of invasiveness may further be examined by inhibition of cell migration. A variety of 2-dimensional and 3-dimensional cellular matrices are commercially available (Calbiochem-Novabiochem Corp., San Diego, Calif.). Cell migration across or into a matrix may be examined by microscopy, time-lapsed photography or videography, or by any method in the art allowing measurement of cellular migration. In a related embodiment, loss of invasiveness is examined by response to hepatocyte growth factor (HGF). HGF-induced cell scattering is correlated with invasiveness of cells such as Madin-Darby canine kidney (MDCK) cells. This assay identifies a cell population that has lost cell scattering activity in response to HGF (Hordijk et al., 1997, Science 278:1464-66).

Alternatively, loss of invasiveness may be measured by cell migration through a chemotaxis chamber (Neuroprobe/Precision Biochemicals Inc., Vancouver, BC). In such assay, a chemo-attractant agent is incubated on one side of the chamber (e.g., the bottom chamber) and cells are plated on a filter separating the opposite side (e.g., the top chamber). In order for cells to pass from the top chamber to the bottom chamber, the cells must actively migrate through small pores in the filter. Checkerboard analysis of the number of cells that have migrated may then be correlated with invasiveness (see, e.g., Ohnishi, 1993, Biochem. Biophys. Res. Commun. 193:518-25).

Identified compounds can also be demonstrated to inhibit tumor formation in vivo. A vast number of animal models of hyperproliferative disorders, including tumorigenesis and metastatic spread, are known in the art (see Table 317-1, Chapter 317, “Principals of Neoplasia,” in Harrison's Principals of Internal Medicine. 13th Edition, Isselbacher et al., eds., McGraw-Hill, New York, p. 1814, and Lovejoy et al., 1997, J. Pathol. 181:130-135). Specific examples include for lung cancer, transplantation of tumor nodules into rats (Wang et al., 1997, Ann. Thorac. Surg. 64:216-219) or establishment of lung cancer metastases in SCID mice depleted of NK cells (Yono and Sone, 1997, Gan To Kagaku Ryoho 24:489-494); for colon cancer, colon cancer transplantation of human colon cancer cells into nude mice (Gutman and Fidler, 1995, World J. Surg. 19:226-234), the cotton top tamarind model of human ulcerative colitis (Warren, 1996, Aliment. Pharmacol. Ther. 10 Supp 12:45-47) and mouse models with mutations of the adenomatous polyposis tumor suppressor (Polakis, 1997, Biochim. Biophys. Acta 1332:F127-F147); for breast cancer, transgenic models of breast cancer (Dankort and Muller, 1996, Cancer Treat. Res. 83:71-88; Amundadittir et al., 1996, Breast Cancer Res. Treat. 39:119-135) and chemical induction of tumors in rats (Russo and Russo, 1996, Breast Cancer Res. Treat. 39:7-20); for prostate cancer, chemically-induced and transgenic rodent models, and human xenograft models (Royai et al., 1996, Semin. Oncol. 23:35-40); for genitourinary cancers, induced bladder neoplasm in rats and mice (Oyasu, 1995, Food Chem. Toxicol 33:747-755) and xenografts of human transitional cell carcinomas into nude rats (Jarrett et al., 1995, J. Endourol. 9:1-7); and for hematopoietic cancers, transplanted allogeneic marrow in animals (Appelbaum, 1997, Leukemia 11 (Suppl. 4):S15-S17). Further, general animal models applicable to many types of cancer have been described, including, but not restricted to, the p53-deficient mouse model (Donehower, 1996, Semin. Cancer Biol. 7:269-278), the Min mouse (Shoemaker et al., 1997, Biochem. Biophys. Acta, 1332:F25-F48), and immune responses to tumors in rat (Frey, 1997, Methods, 12:173-188).

For example, an identified compound can be administered to a test animal, in one embodiment a test animal predisposed to develop a type of tumor, and the test animal subsequently examined for an decreased incidence of tumor formation in comparison with an animal not administered the identified compound. Alternatively, a identified compound can be administered to test animals having tumors (e.g., animals in which tumors have been induced by introduction of malignant, neoplastic, or transformed cells, or by administration of a carcinogen) and subsequently examining the tumors in the test animals for tumor regression in comparison to animals not administered the identified compound.

5.3 Kits

This invention encompasses kits which can simplify the performance of assay methods of the invention. A typical kit of the invention comprises individual components of the assay, preferably in unit concentration. Such components include, but are not limited to: p27 and Cdk2/Cyclin E, individually or as a complex; polypeptides used in assay methods of this invention, i.e., E1, E2, and E3 (as individual constituents or as a complex); ubiquitin, optionally labeled; plates, optionally with appropriate coatings on surfaces (e.g., protein A or protein G-coated plates, preferably black); labeling material; coating material; and buffers and other reagents required for the assay. In a specific embodiment, said kit comprises an antibody to p27, an antibody to a protein of interest, or an antibody to ubiquitin. Where the kit provides materials for the FRET assay, the kit, in one embodiment, provides a donor fluorophore and an acceptor fluorophore. In a specific embodiment, said donor fluorophore is in a form that binds to p27 (e.g., phosphorylated p27). In another embodiment, said acceptor fluorophore is in a form that binds ubiquitin.

Each component may be individually packaged, or where appropriate, two or more components can be packaged together. In some embodiments, recombinant DNAs (e.g., vectors) that encode the components of the assay can be included instead of purified components themselves. In such cases, materials required for expression and purification of the components may be also included in the kits. Examples of such materials include, but are not limited to, host cells, medium components, and purification apparatus and reagents for, among others, tagged molecules.

The invention is further defined by reference to the following non-limiting examples. It will be apparent to those skilled in the art that many modifications, both to materials and methods, can be practiced without departing from the spirit and scope of this invention.

### 6. EXAMPLES

### 6.1 Example 1

### p27 Ubiquitination Assay—Plate Capture Format

This assay measures the ubiquitination of cell cycle inhibitor p27 in a high-throughput-capable in vitro reconstituted system that mimics the ubiquitination of p27 in vivo. Ubiquitinated p27 is captured using an anti-p27 antibody-coated plate and detected by Europium (Eu)-labeled streptavidin that binds biotinylated ubiquitin. The assay as described below incorporates the addition of a test compound to the ubiquitination reaction.

The assay rests on at least the following protein interactions. E1 activates ubiquitin in the presence of Mg⁺⁺ and ATP, forming a thioester bond between the C-terminus of ubiquitin and an active cysteine of E1. Activated ubiquitin is then transferred to E2. In the presence of the E3 complex and Cks1, ubiquitin carried by E2 is transferred to p27, forming an isopeptide bond between the C-terminus of ubiquitin and the side chain of lysine residues on p27. Poly-ubiquitin chains are formed by isopeptide bonds between the C-terminus of ubiquitin and the side chain of a lysine residue another ubiquitin molecule.

6.1.1 Materials and Methods

E1, E2 and Cks1: His-E1, E2 (His-UBcH3) and Strep-tagged Cks1 was expressed in Escherichia coli and purified on either a nickel chelate column or S column (for Cks1). Cks1 was removed from the S column using thrombin cleavage. The recovered proteins were dialyzed against a ubiquitin buffer (30 mM Tris-HCl pH 7.5, 20% glycerol, 1 mM DTT) and stored in aliquots at −80° C.

E3: Insect cells were co-infected with baculoviruses expressing GST-Skp2, Skp1, Cul1 (Cullin), and Roc1 individually. The resulting SCF^{Skp2} complex was purified using glutathione agarose beads. After dialysis in ubiquitin buffer, the complex was stored in aliquots at −80° C.

Biotin-ubiquitin: 100 mg ubiquitin (Sigma U6253) was dissolved in 10 phosphate buffered saline (PBS). 12.5 mg EZ-link™ Sulfo-NHS-LC-Biotin (Pierce Biotechnology, Catalog #21335) was added and incubated on ice for two hours.

p27/Cdk2/Cyc E: Human p27 was co-expressed with Cdk2 and His-tagged cyclin E in Sf9 cells. The p27/Strep-Cdk2/His-Cyclin E was purified through a nickel chelate column and dialyzed into ubiquitin buffer.

Cdk2/Cyc E: Cdk2/His-Cyclin E was prepared in the same manner as p27/Cdk2/His-Cyclin E.

Pre-phosphorylated p27 was prepared by incubating 0.1 mg/ml Cdk2/Cyc E with 0.1 mg/ml p27/Cdk2/Cyc E at room temperature for 2 h in kinase buffer (40 mM Tris-HCl pH 7.5, 10 mM MgCl₂, 1 mM DTT, 1 mM ATP).

Other components/materials: 384-well protein A-coated black plates were custom-made by Pierce Biotechnology. sc-528, a rabbit polyclonal antibody raised against a C-terminal peptide of p27 (C-19), was obtained from Santa Cruz Biotechnologies (Santa Cruz, Calif.). The assay also used Eu-Streptavidin (Perkin Elmer #1244-360; 0.1 mg/ml), enhancement solution (Perkin Elmer #1244-105), assay diluent (BD Pharmingen #555213, phosphate buffered saline, and a wash buffer of 10 mM Tris-HCl pH 7.5, 0.05% (v/v) Tween 20.

Assay mix A (see below) contained: 3× concentration ubiquitin buffer (120 mM Tris·HCl pH 7.5, 15 mM MgCl₂), 3 mM dithiothreitol, 15 ng/μL E1, 450 ng/μLE2, 15 ng/μL E3, and 12 ng/μL pre-phosphorylated p27.

Starting solution (see below) contained 0.75 mg/mL biotinylated ubiquitin and 0.75 ng/μL Cks1 in assay dilution buffer (30 mM Tris·HCl pH7.5, 1 mM DTT, 19.5% glycerol). Starting solution for negative control contains 0.75 mg/mL biotinylated ubiquitin in assay dilution buffer (30 mM Tris·HCl pH7.5, 1 mM DTT, 19.5% glycerol).

6.1.2 Assay Procedure

The capture plates were coated as follows. Wells of the protein A-coated plates were washed 3 times with PBS containing 0.05% (v/v) Tween 20. To each well was added 25 μL of 2.5 μg/ml sc-528 antibody diluted in PBS. The antibody and plate were allowed to incubate overnight at room temperature. The next day, unbound antibody was discarded. 100 μL assay diluent was added to each well and incubated for 1-4 hours at room temperature. The assay diluent was discarded just prior to adding the reaction mixture to each well.

The ubiquitination reaction was run as follows: 5 μL of a test compound in 6% dimethylsulfoxide (DMSO) was added to each well of a V-bottom plate. To each well was added 5 μL assay mix A and 5 μL starting solution (experimental conditions) or starting solution without Cks1 (control conditions). This mixture was incubated at room temperature for 45 minutes, and 20 μL assay diluent was added per well. 20 μL of this mixture in each well was transferred to the coated protein A capture plates described above and incubated for 1 hour at room temperature. The wells were then washed 6 times with wash buffer (10 mM Tris-HCl pH 7.6, 0.05% (v/v) Tween 20). After washing, 25 μl of Eu-streptavidin at a final concentration of 0.4 μg/ml in 25 mM Hepes pH 7.6, 1% BSA, 0.2% Tween 20 was added to each well and incubated for 1 hour at room temperature. The wells were then washed 6 times with wash buffer (10 mM Tris-HCl pH 7.6, 0.05% (v/v) Tween 20). 25 μL enhancement solution was added per well, and the plates were read to determine the amount of time-resolved fluorescence emission at 615 nm for Europium chelate.

### 6.2 Example 2

### Assay Optimization

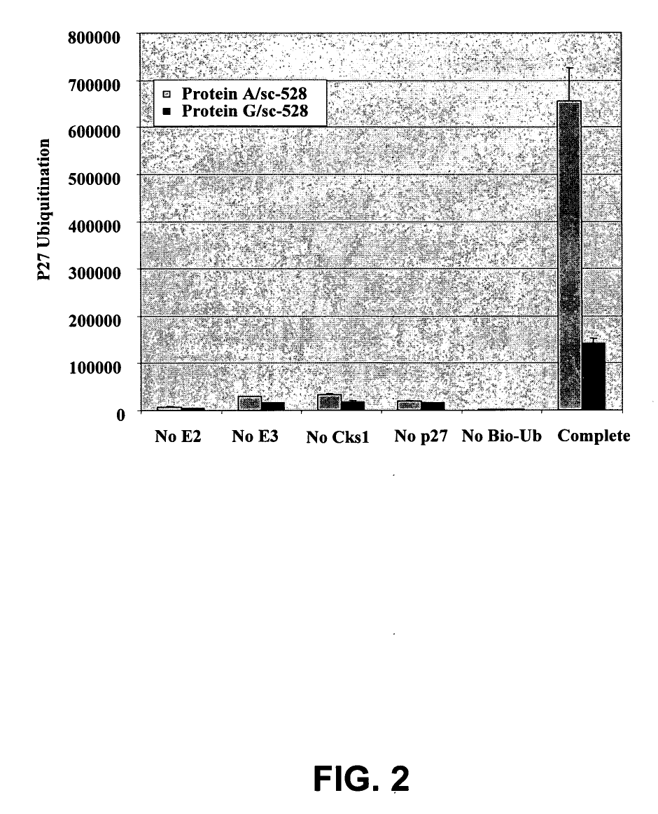

The signal captured on either protein A or protein G plates is dependent upon pre-phosphorylated p27, E2, E3, Cks1 and biotinylated ubiquitin, indicating that this assay is specific for p27 ubiquitination. Mixtures lacking any of these components failed to produce a significant signal in the assay (FIG. 2).

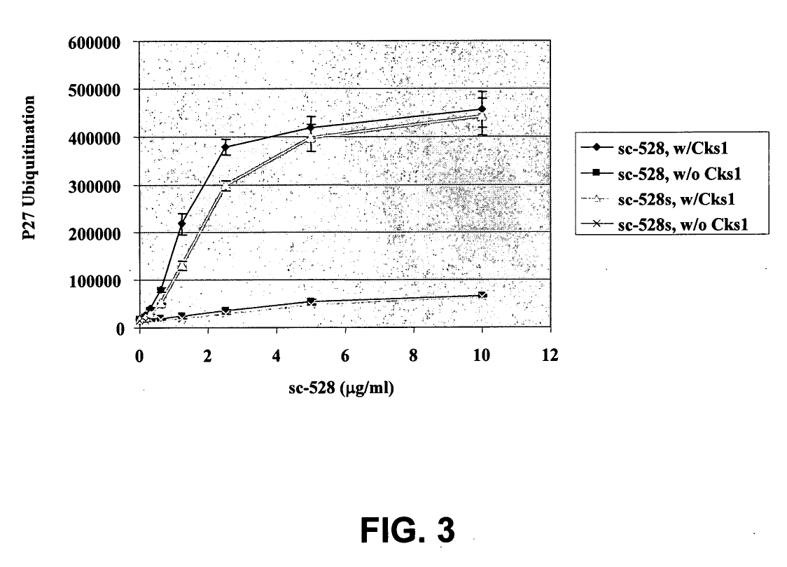

For assay optimization, the assay was performed essentially as described above. Two different lots of sc-528 antibody were diluted serially and coated on a protein A-coated capture plate. Pre-phosphorylated p27 (4 ng/μL) was completely ubiquitinated and was captured on the plate and detected with Eu-Strep (1:1000) (FIG. 3A). The optimum amount of sc-528 in the assay was determined to be 2.5 μg/mL; this concentration was chosen for the standard assay condition.

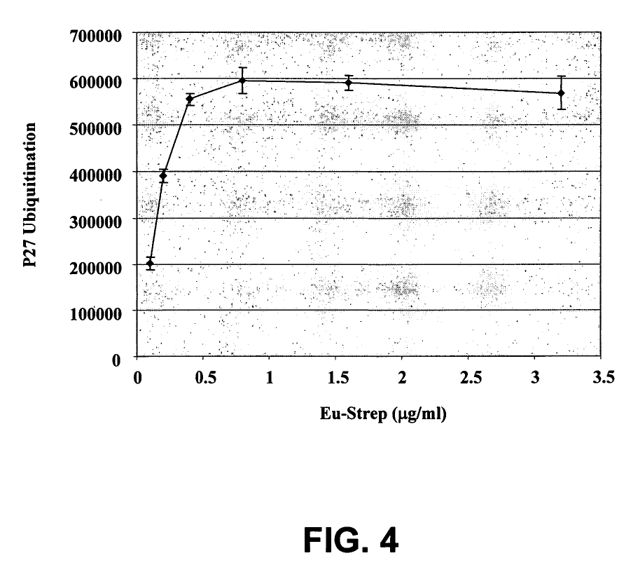

For titration of Eu-Streptavidin, Pre-phosphorylated p27 (4 ng/μL) was completely ubiquitinated and was captured on a protein A plate coated with 2.5 μg/ml antibody sc-528. Eu-Strep (0.1 mg/ml) was diluted serially and used to detect ubiquitinated p27. The optimal amount of Eu-Streptavidin to be used for each reaction was determined to be 0.4 μg/ml (1:250 dilution; FIG. 4). This concentration was chosen for the standard assay condition.

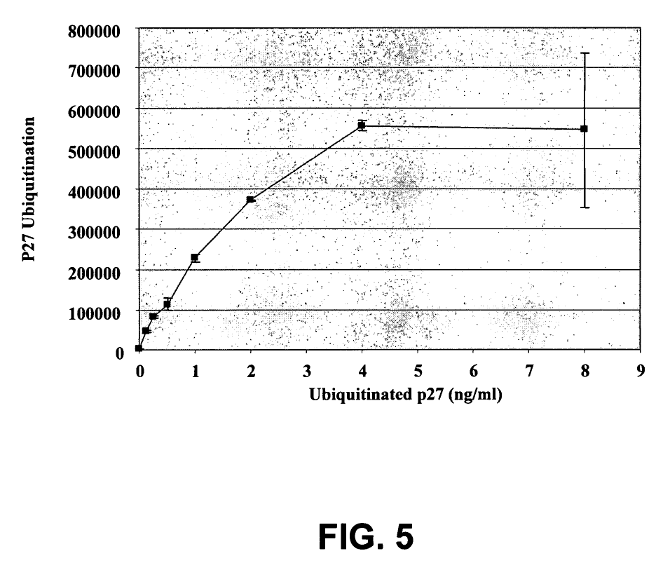

For titration of ubiquitinated p27, capture antibody and Eu-Streptavidin was used at the concentrations determined above. pre-phosphorylated p27 (8 ng/μL) was completely ubiquitinated and titrated. Eu-Strep fluorescence was determined to be linear up to 4 ng/μL pre-phosphorylated p27 (FIG. 5); this concentration was chosen as the pre-phosphorylated p27 concentration for the assay. Under standard assay conditions as indicated in Table 2, approximately 50% of pre-phosphorylated p27 was ubiquitinated.

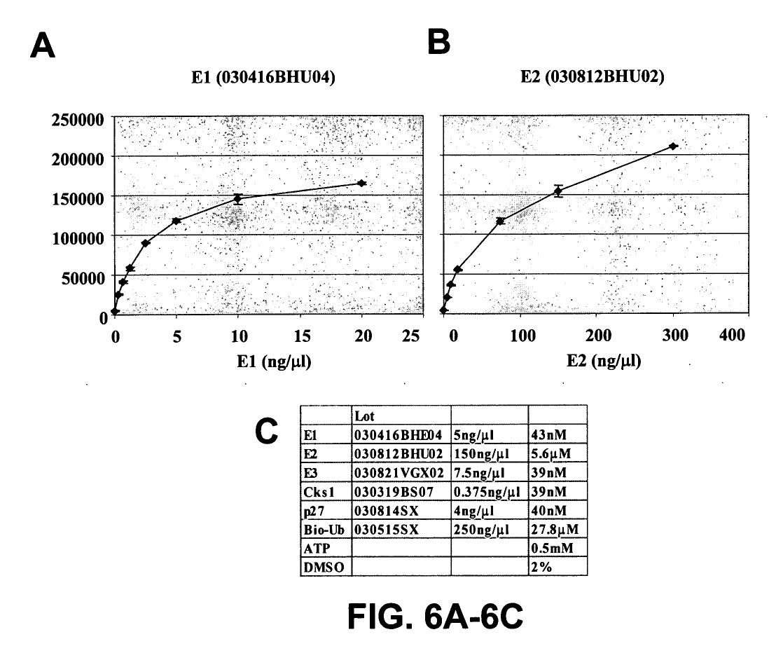

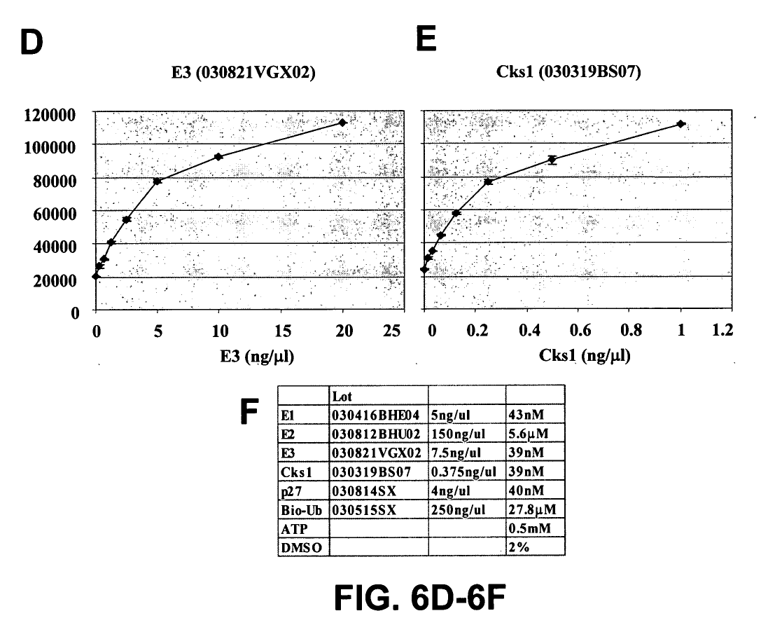

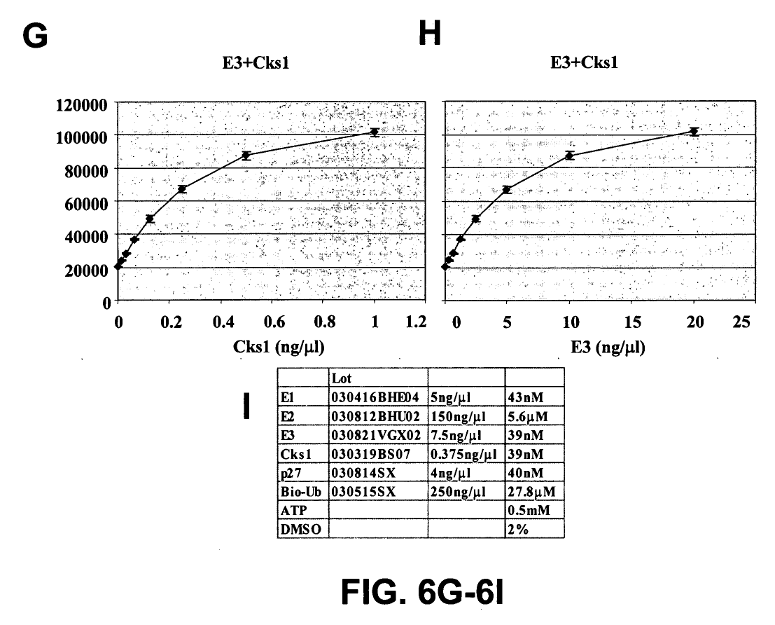

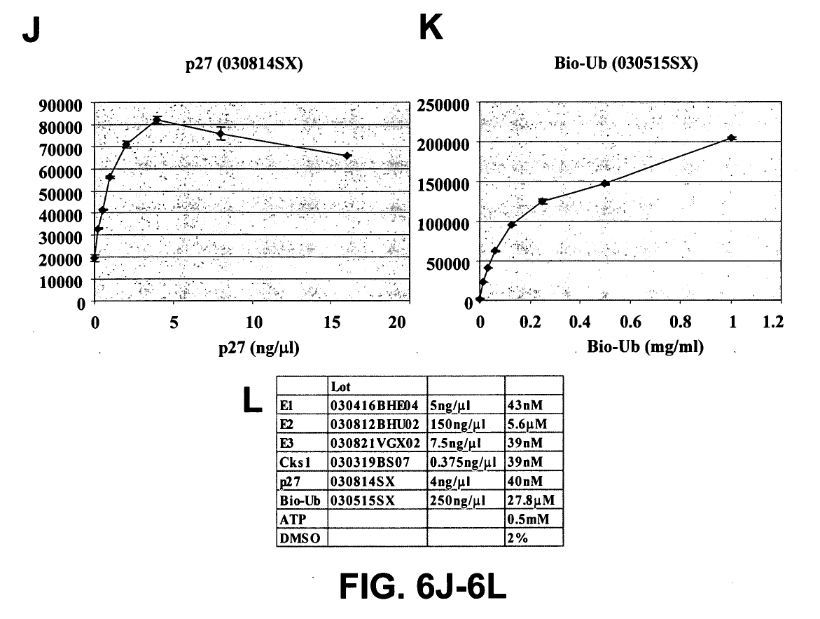

A matrix titration between Cks1 and E3, E3 and E2, E2 and E1, and titration of biotinylated ubiquitin were performed. Under ideal conditions, E1, E2, pre-phosphorylated p27 and biotinylated ubiquitin should be used at saturating conditions. However, E2 could not be saturated at a concentration as high as 300 ng/μL. Therefore, 150 ng/μL was chosen as the concentration for E2. For titration of each of E1, E2, E3, Cks1, pre-phosphorylated p27, biotinylated ubiquitin, and E3:Cks1 (FIGS. 6A-6J), the remaining components of the reaction mixture were held constant at the concentrations shown in Table 2.

TABLE 2
Concentration of constant components within each
titration experiment for E1, E2, E3 and Cks1

| | | | | |
|---|---|---|---|---|
| E1 | 5 | ng/μL | 43 | nM |
| E2 | 150 | ng/μL | 5.6 | μM |
| E3 | 7.5 | ng/μL | 39 | nM |
| Cks1 | 0.375 | ng/μL | 39 | nM |
| Pre-phosphorylated p27 | 4 | ng/μL | 40 | nM |
| Biotinylated ubiquitin | 250 | ng/μL | 27.8 | nM |
| Tris.HCl, pH 7.5 | | | 40 | mM |
| MgCl₂ | | | 5 | mM |
| DTT | | | 1 | mM |
| ATP | | | 0.5 | mM |

| | |
|---|---|
| DMSO | 2% |

The optimal concentration of these components was determined to be: E1-5 ng/μL (43 nM); E2-150 ng/μL (5.6 μM); E3-5 ng/μL (26 nM); and Cks1-0.25 ng/μL (26 nM).

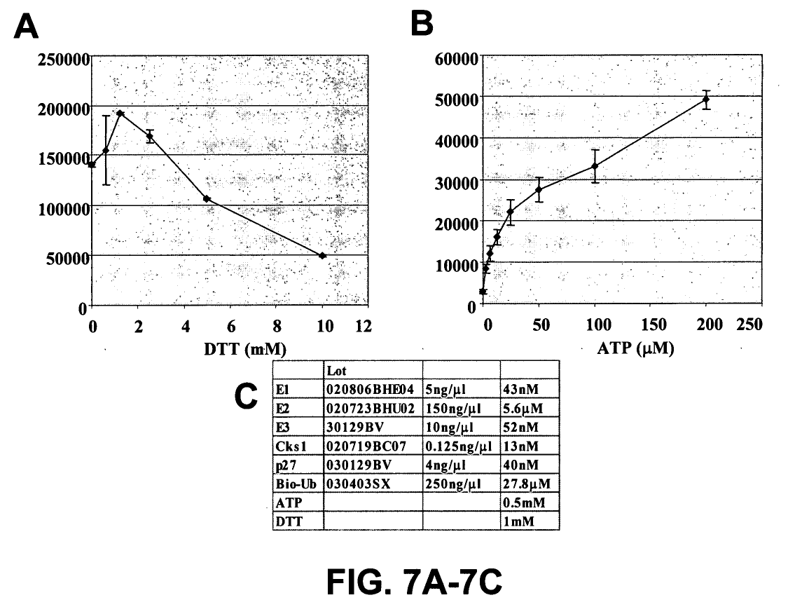

Titration of ATP and DTT: For ATP titration, p27/Cdk2/Cyc E and Cdk2/Cyc E were added to the reaction separately because the pre-phosphorylated p27 contained ATP derived from the phosphorylation assay. Therefore, two ATP-dependent reactions exist: (1) the phosphorylation of p27 by Cdk1/Cy E; and (2) the activation biotinylated ubiquitin by E1. A presumably saturating amount of ATP (500 μM) was selected as optimal (FIG. 7A). For DTT, 1 mM was selected as optimal (FIG. 7B).

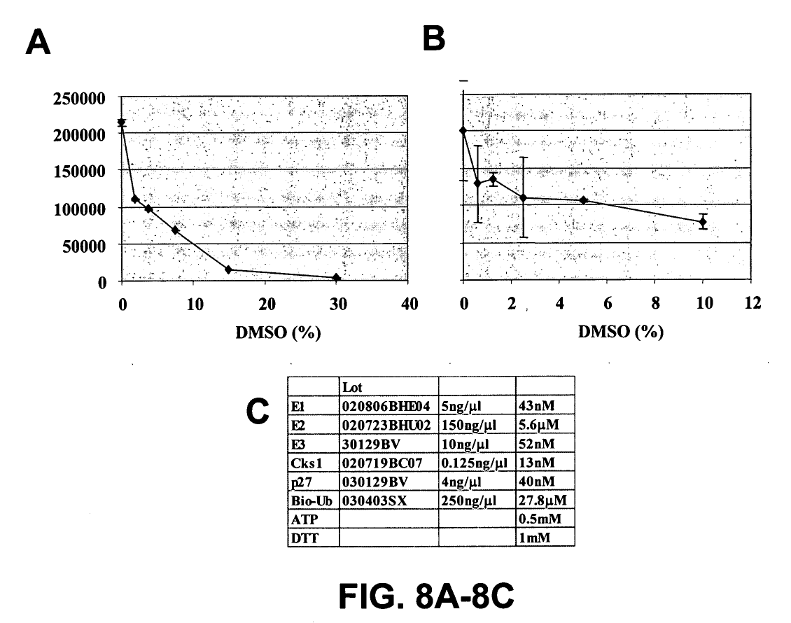

Determination of DMSO tolerance was as follows. p27 ubiquitination is sensitive to DMSO. An initial titration (FIG. 8A) showed that p27 ubiquitination was nearly completely lost above 15% (v/v) of DMSO. A second titration of concentrations between 0 and 10% DMSO (FIG. 6B) showed that approximately 60% of the p27 ubiquitination activity remained at 2% DMSO; a 2% final concentration of DMSO was thus selected.

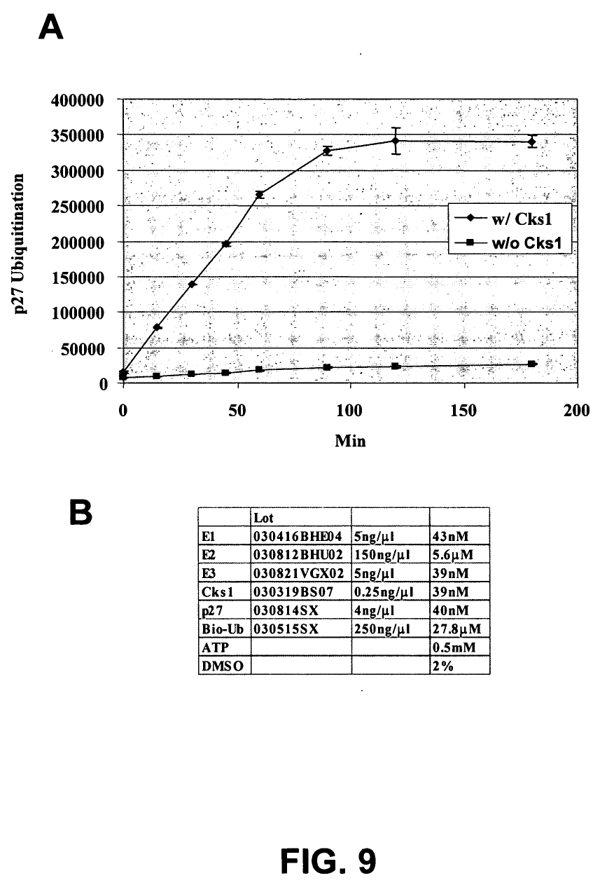

Time course: pre-phosphorylated p27 was ubiquitinated for the time indicated in FIG. 9 in the presence of the components listed in Table 1, above. The optimized assay is linear for up to 1.5 hours. An incubation within this period, 45 minutes, was selected. At this incubation time, the results of the optimized assay correlated well with p27 ubiquitination of the same samples as detected by gel in a western blot.

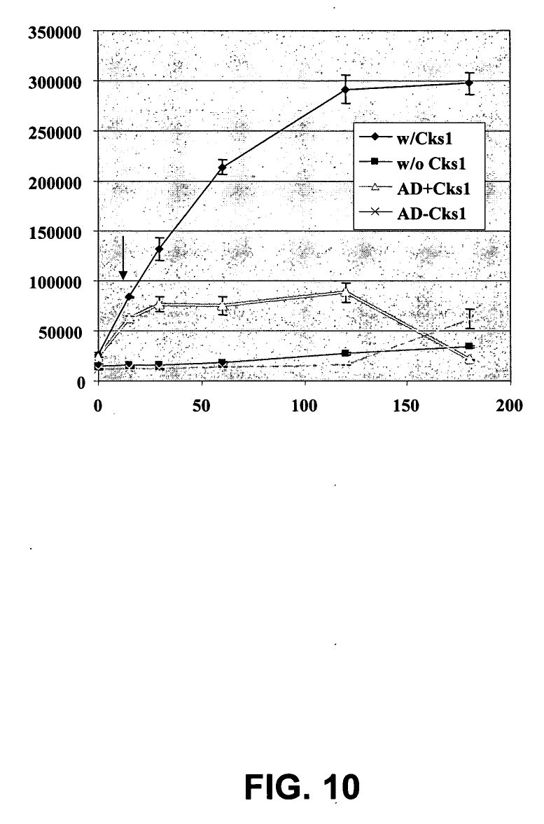

Stopping the reaction could be accomplished in several different ways. The method selected was the addition of assay diluent (FIG. 10).

### 6.3 Example 3

### Computer-Implemented High-Throughput Screen for Compounds that Modulate p27 Ubiquitination

This Example describes modifications of the assay described in Example 1 for use in a high-throughput screen of compounds that increase or decrease the ubiquitination of p27.

Test compounds generated as part of a combinatorial chemistry library are tested for the ability to modulate p27 ubiquitination using the assay described in Example 1. The amount of ubiquitination (i.e., the fluorescence at 615 nm) is compared to the fluorescence of the control condition (i.e., the same reaction performed substituting 5 μL 6% DMSO for the test compound solution). Compounds are identified as modulating the ubiquitination of p27 if the fluorescence in a test compound condition deviates from the fluorescence of the control condition by 50%. Compounds that are identified as modulating the ubiquitination of p27 are tested again using a statistically significant sample size (e.g., ten parallel p27 ubiquitination reactions using the same test compound in each). Compounds confirmed as showing a statistically significant modulation of p27 ubiquitination are serially diluted by half for 5-10 successive concentration conditions (i.e., 1×, 0.5×, 0.25×, 0.125×, etc.) to determine ED₅₀ concentrations.

### 6.4 Example 4

### p27 Homogenous Time-Resolved Fluorescence Resonance Energy Transfer (HTR-FRET) Assay Format

A homogenous, time-resolved fluorescence resonance energy transfer (HTR-FRET) assay was developed for high throughput screening of compounds that block p27 ubiquitination in vitro. In this assay, p27 is modified with a mixture of ubiquitin (Ub) and biotinylated ubiquitin (Bio-Ub) in the presence of E1, E2, E3 and Cks1 (FIG. 11). A rabbit anti-phospho-p27 antibody and Lance Eu labeled Protein G (Eu-PRG) were used as a donor and Cy5-labeled streptavidin (Cy5-SA or Dylite-SA) is used as an acceptor. Ubiquitinated p27 is detected by FRET signal generated between Eu and Cy5. This homogenous p27 Ub assay has a very simple procedure consisting only of four addition steps, and no separation steps, prior to reading the results of the reaction. The simplicity of the assay procedure reduces the assay time, increasing throughput, and provides high quality data with good Z′ statistics.

6.4.1 Materials and Methods

Preparation of Protein Components
1) E1, E2, and Cks1: His-E1 was expressed from baculovirus-infected Sf9 cells. His-UbcH3 and the Strep tagged Cks1 were expressed in E. coli. His-E1, His-UbcH3, and Strep tagged Cks1 were purified by either Ni²⁺ chelate or anti-Strep tag chromatography, respectively. The Strep tag was removed from Cks1 by thrombin cleavage and the cleaved tag removed by anti-Strep tag chromatography. The proteins were dialyzed against Ub buffer (30 mM Tris-HCl pH 7.5, 20% glycerol, 1 mM DTT) and stored in small aliquots at −80° C.
2) E3: Insect cells were co-infected with baculoviruses expressing GST-Skp2, His-Skp1, His-Cul1, and Roc1 individually. The SCF^{Skp2} complex was purified on glutathione agarose beads. After dialysis in Ub buffer, the protein complex was stored in small aliquots at −80° C.
3) Bio-Ub: 100 mg of Ub (Sigma U6253) was dissolved in 10 ml PBS. 12.5 mg of EZ-link™ Sulfo-NHS-LC-Biotin (Pierce Biotechnology, Catalog #21335) was added. After incubation on ice for 2 h, the labeled ubiquitin was dialyzed into 10 mM Hepes pH8.0 and stored in small aliquots at −80° C.
4) Ub: ubiquitin was purchased from Sigma (U6253) and dissolved in H₂O or PBS at 10 mg/ml.
5) P27/Cdk2/Cyc E: Human p27 was co-expressed with Strep-tagged Cdk2 and His tagged cyclin E in Sf9 cells. The p27/Strep-Cdk2/His-Cyclin E was purified via Ni²⁺ chelate chromatography and dialyzed into Ub buffer
6) Cdk2/Cyc E: Strep-Cdk2/His-Cyclin E was prepared in a similar way to p27/Strep-Cdk2/His-Cyclin E.
7) Phospho p27 was prepared by incubating 0.1 mg/ml Cdk2/Cyc E with 0.1 mg/ml p27/Cdk2/Cyc E at room temperature for 2 h in kinase buffer (40 mM Tris-HCl, pH7.5, mM MgCl₂, 1 mM DTT, 1 mM ATP)

Other Materials:
1) Assay plates: Greiner 384-well black polypropylene plate, cat# 3710
2) Rabbit anti-phospho-p27: Zymed (Cat# 71-7700); 0.25 mg/ml.
3) Eu-Protein G: Perkin Elmer (AD0071), 0.4 mg/ml (19.1 μM).
4) SA-Cy5: Amersham (PA92005V), 1 mg/ml in H₂O.
5) SA-Dylite: Pierce Biotechnology (21824), 1 mg/ml
6) 10×Ub buffer: 400 mM Tris HCl pH7.5, 50 mM MgCl₂.
7) Assay dilution buffer: 30 mM Tris HCl pH7.5, 1 mM DTT, 19.5% glycerol, 0.03% Brij 35; stored at 4° C.

Reagent Handling

All the protein reagents were divided into aliquots (each aliquot equal to the amount needed for screening forty, 384-well plates) and stored at −80° C. The proteins need to be kept on ice at all times when in use and the remaining portion should be frozen on dry ice and stored at −80° C. Frequent freeze/thaw cycles should be avoided.

6.4.2 Assay Development

After comparing mono-ubiquitination with polyubiquitination, different anti-p27 antibodies and secondary antibodies, different donor/acceptor pairs, etc., a HTR-FRET assay format as illustrated in FIG. 11 was chosen. In this format, Bio-Ub is used as a tracer to label ubiquitinated p27. Lance Eu-labeled Protein G (Eu-PRG) coupled to anti-phospho-p27 is used as the FRET donor, and SA-Cy5 is used as the FRET acceptor. The FRET signal is measured as the ratio of fluorescence emission of Cy5 at 665 nm over that of Eu at 620 nm after excitation of Eu at 340 nm.

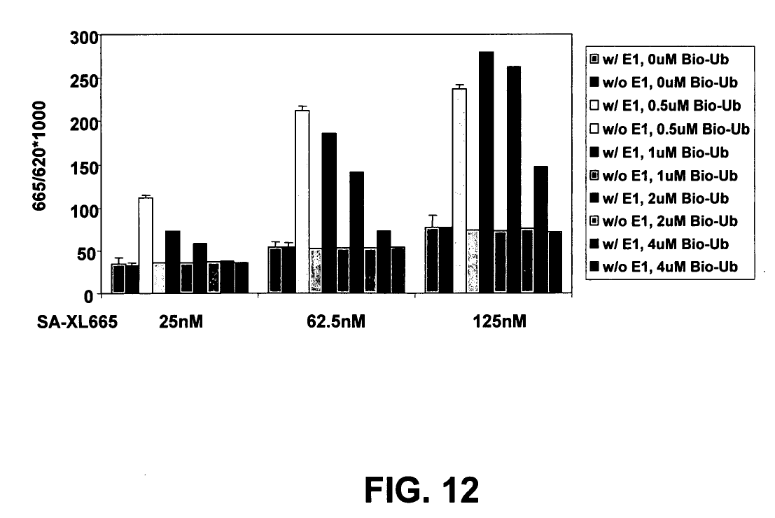

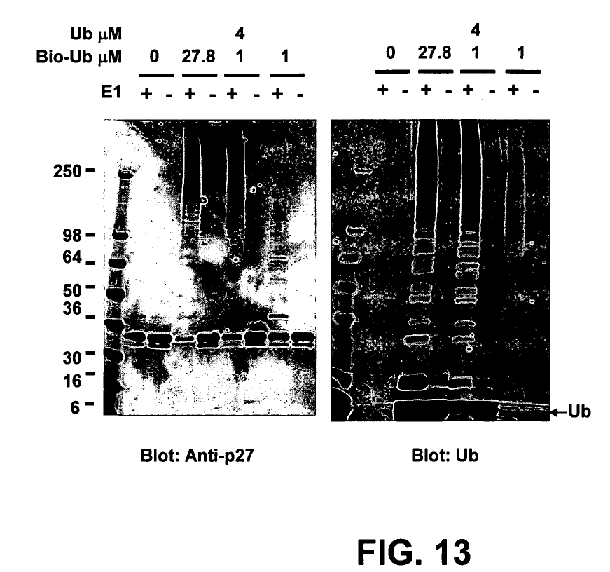

In an HTR-FRET assay, the background FRET increases as the concentrations of Eu and Cy5 increase. In general, to achieve a low background signal, Eu concentrations should be in the low nM range while Cy5 concentrations can be up to 100-200 nM. To determine the optimal concentration of Bio-Ub used in the p27 Ub assay, 80 nM of p27 was ubiquitinated with 0-4 μM of Bio-Ub and diluted 1:4 with a detection mixture containing a final concentration of 5 nM Eu-PRG, 10 nM anti-p27, and 25-125 nM SA-Cy5. 1 uM of Bio-Ub with 125 nM of Cy5-SA gave the best signal-to-background (S/B) ratio (FIG. 12). Further increasing the concentration of Cy5-SA increased the background without significantly increasing the signal (data not shown). Since the apparent Kₘ of Ub for E1 is about 2 μM, 4 μM of unlabeled ubiquitin was used in the reaction (2×apparent Kₘ). Ubiquitination of p27 in the presence of 4 μM Ub and 1 μM Bio-Ub was very similar to that of p27 in the presence of saturating concentrations of Bio-Ub (28 μM) (FIG. 13). Ubiquitination of p27 in the presence of 1 μM Bio-Ub alone was much lower, which suggests that Bio-Ub may become limiting at 1 μM of total ubiquitin in the reaction.

Next, stopping conditions for the p27 ubiquitination were determined. Ubiquitination of p27 was completely inhibited by 20 mM EDTA (data not shown). Although the reaction includes only 5 mM MgCl₂ (Table 2), 5 mM EDTA alone had no effect on p27 ubiquitination, and 10 mM EDTA only partially inhibited p27 ubiquitination. The ubiquitination of p27 detected by the HTR-FRET assay correlated well with that detected by western blot, suggesting that the signal detected by HTR-FRET faithfully reflects the ubiquitination of p27.

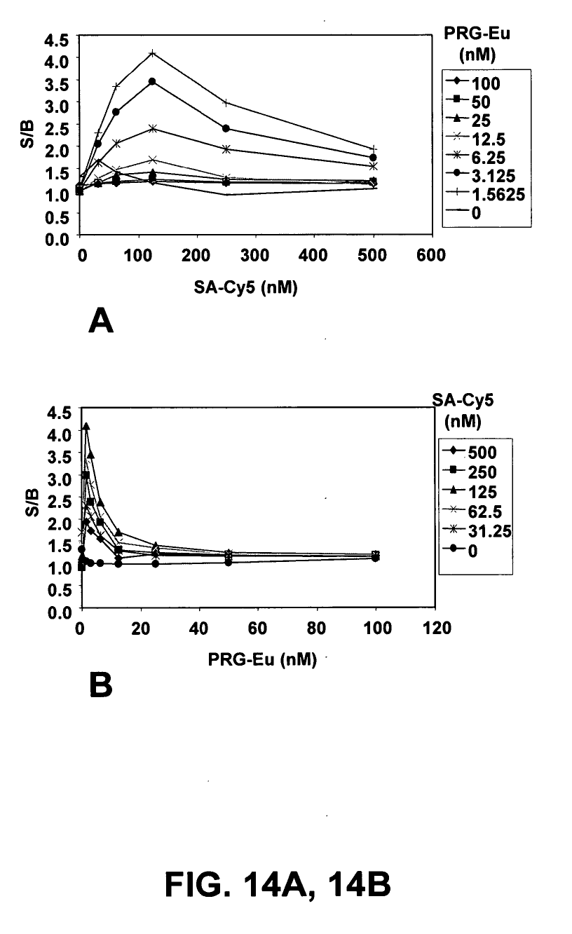

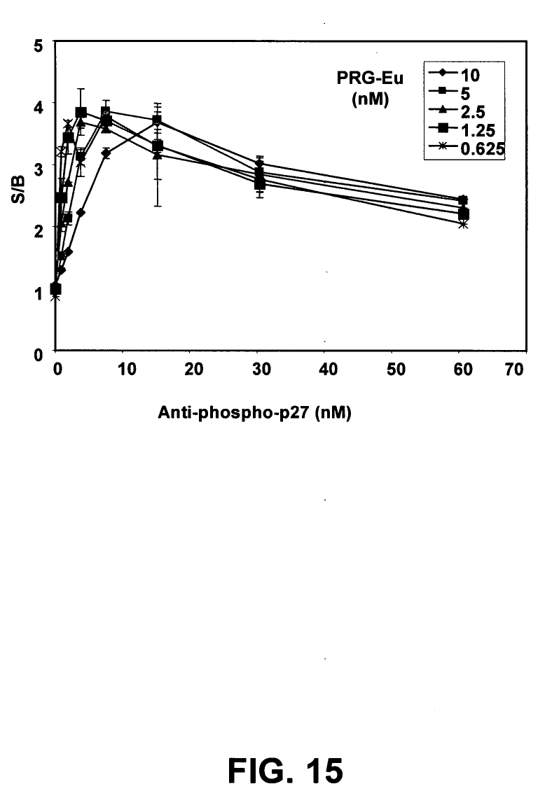

To optimize the concentrations of SA-Cy5 and Eu-PRG, 40 nM of p27 was ubiquitinated with 4 μM Ub and 1 μM Bio-Ub. The reaction was stopped by 1:2 dilution with a stop solution containing 40 mM EDTA, 6.67 nM anti-phospho p27, and various concentrations of Cy5-SA (FIG. 14A) and Eu-PRG (FIG. 14B). The best S/B was achieved with 125 nM Cy5-SA and ˜2 nM Eu-PRG. Higher concentrations of either detection reagent decreased S/B. With Cy5-SA at 125 nM, anti-phospho p27 and Eu-PRG were titrated in a similar manner (FIG. 15). The optimal concentrations for anti-phospho p27 and Eu-PRG were 3.75 nM and 1.25 nM, respectively.

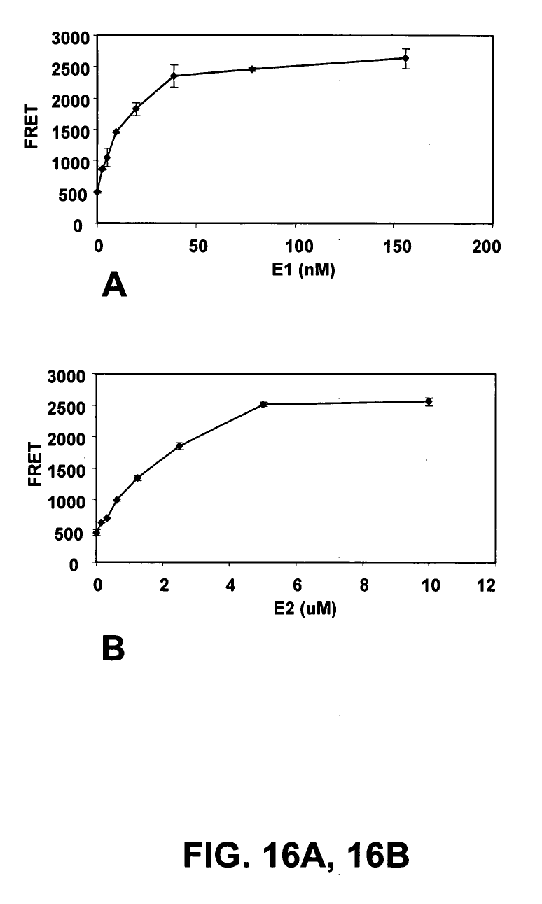

Titration of E1 and E2 (FIGS. 16A and 16B, respectively) suggested that E1 and E2 became saturating at 5 ng/μl (39 nM) and 150 ng/μl (5 μM), respectively, consistent with what we observed in the plate capture format p27 Ub assay (see Examples 1 and 2, above). 5 ng/μl (39 nM) of E1 and 150 ng/μl (5 μM) of E2 were chosen.

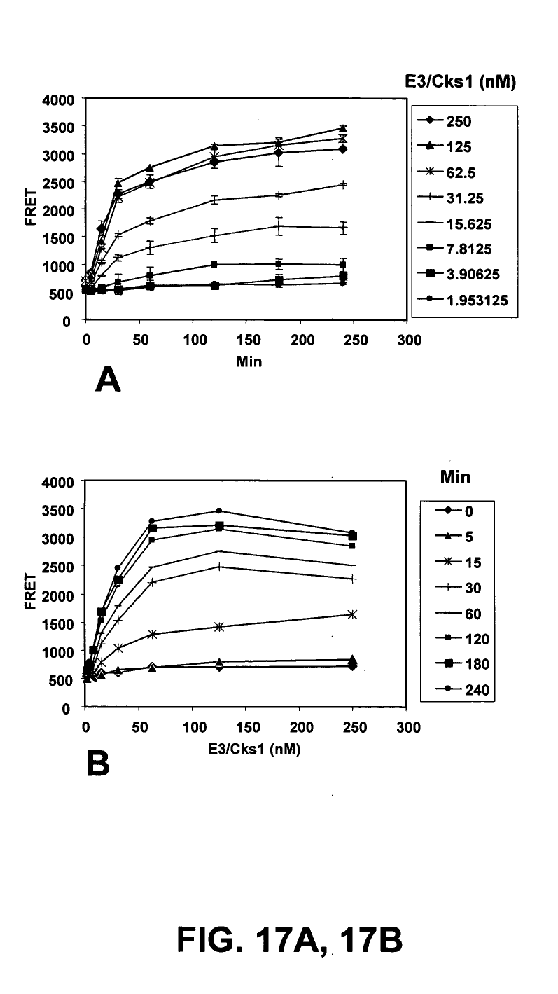

FIGS. 17A and 17B show a titration of E3 and Cks1 and time dependence of the reaction at various E3/Cks 1 concentrations. Under the experimental conditions, the maximal FRET signal was 3500. The E3 and Cks1 at 62.5 nM and an incubation time of 1 h gave ˜70% of the maximal signal; these concentrations were chosen for screening.

Based on the above data, a final protocol for the p27 ubiquitination assay in HTR-FRET format was established.

Assay Procedures:
1) Prepare Starting solutions and incubate at room temperature for 20˜30 min Start+Cks1: 12 μM Ub, 3 μM Bio-Ub, and 1.875 ng/μl (187.5 nM) Cks1 in assay dilution buffer. Start−Cks1: 12 μM Ub and 3 μM Bio-Ub in assay dilution buffer.
2) Prepare Assay mix A as indicated in Table 3
3) Set up reaction: In an assay plate, add 5 μl/well of Assay mix A, compound in 6% DMSO and 0.03% Brij35 (30 μg/ml; final concentration at 10 μg/ml), and Starting solutions. The total assay volume is 15 μl. Incubate at room temperature (˜23° C.-25° C.) for 1 hour.
4) Stop the reaction with the addition of 15 μl/well of Stopping solution (40 mM EDTA, 0.1% Tween 20, 250 nM Cy5, 2 nM Eu-PRG, and 8 nM anti-phospho-p27 in PBS)

5) Incubate at RT for 1-2 h before reading the plate on an Analyst HT plate reader (Molecular Device).

TABLE 2
Assay components

| Components | Reagent Name | Concentration | Source | Lot |
|---|---|---|---|---|
| E1 | His-E1 | 5 ng/μl (39 nM) | Baculovirus | 050107VHE04 |
| E2 | His-UbcH3 | 150 ng/μl (5 μM) | E. coli | 050111BHU02 |
| E3 (SCF^{Skp2}) | GST-E3 | 12.5 ng/μl (62.5 nM) | Baculovirus | 050107VGE04 |
| Cks1 | Cks1 | 0.625 ng/μl (62.5 nM) | E. coli | 050111BC07 |
| Substrate | Phospho p27 | 4 ng/μl (40 nM) | Baculovirus | 031218SX |
| Biotinylated | Bio-Ub | 9.1 ng/μl (1 μM) | Sigma | 041208SXPP |
| ubiquitin | | | | |
| Ubiquitin | Ub | 34.3 ng/μl (4 μM) | Sigma | |
| Tris-HCl, pH 7.5 | | 40 mM | | |
| MgCl2 | | 5 mM | | |
| DTT | | 1 mM | | |
| ATP | | 0.5 mM | | |
| Brij 35 | | 0.03% | | |
| DMSO | | 2% | | |

TABLE 3
Assay Mix A components.

| | Concentration in | |
|---|---|---|
| Component | Mix A | Final Concentration |
| 10 × Ub buffer | 3× | 1× |
| DTT | 3 mM | 1 mM |
| ATP | 1.5 mM | 0.5 mM |
| E1 | 15 ng/μl | 5 ng/μl (39 nM) |
| E2 | 450 ng/μl | 150 ng/μl (5 μM) |
| E3 | 37.5 ng/μl | 12.5 ng/μl (62.5 nM) |
| Phospho p27 | 12 ng/μl | 4 ng/μl (40 nM) |
| Brij 35 | 0.03% | 0.03% |

Abbreviations:

Ub: ubiquitin; Bio-Ub: biotinylated ubiquitin; Cdk2/Cyc E: Cdk2/Cyclin E; E1: ubiquitin-activating enzyme (Uba); E2: Ubiquitin-conjugating enzyme (Ubc); E3: Ubiquitin ligase; SCF: Skp1, Cul1, and Roc1; RT: room temperature; PBS: phosphate-buffered saline; HTR-FRET: homogenous time-resolved fluorescence resonance energy transfer; Eu-PRG: Lance Eu labeled Protein G; SA-Cy5: Cy5 labeled streptavidin; S/B: signal/background

All of the references cited herein are incorporated by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A method of determining the amount of ubiquitination of a protein, comprising:
a. contacting said protein with a plurality of polypeptides, said plurality of polypeptides together capable of ubiquitinating said protein, to form ubiquitinated protein;
b. capturing said ubiquitinated protein on a surface; and
c. determining an amount of ubiquitin present in said protein captured on said surface, wherein the amount of ubiquitin present in said protein captured on said surface is correlated with the amount of ubiquitination of said protein.

2. The method of claim 1, wherein said protein is p27.

3. The method of claim 1, wherein said plurality of polypeptides are each isolated polypeptides.

4. The method of claim 1, wherein said plurality of polypeptides comprises E1, E2, E3, Cks1 and ubiquitin.

5. The method of claim 2, wherein said E1, E2, E3 or Cks1 is recombinantly produced.

6. The method of claim 2, wherein said E1, E2, E3 and Cks1 are purified from a cell extract.

7. The method of claim 4, wherein said ubiquitin is labeled.

8. The method of claim 7, wherein said label is biotin.

9. The method of claim 7 wherein said labeled ubiquitin is visualized with Europium that is coupled to streptavidin.

10. The method of claim 1, wherein the determination occurs in a multi-well plate as part of a high-throughput screen.

11. A method of identifying a compound that modulates ubiquitination of p27, comprising determining the amount of ubiquitinated p27 formed by combining of isolated phosphorylated p27, E1, E2, E3, Cks1 and ubiquitin in the presence of said compound and in the absence of said compound, wherein, if the amount of ubiquitinated p27 formed in the presence of said compound differs from the amount of ubiquitinated p27 formed in the absence of said compound, said compound is identified as a compound that modulates the ubiquitination of p27.

12. The method of claim 11, wherein said p27 is phosphorylated with Cdk2 and Cyclin E prior to combination with said E1, E2, E3, Cdk2 and ubiquitin.

13. The method of claim 11, wherein said phosphorylated p27 is present at a concentration of about 4 ng/μL; said E1 is present at a concentration of about 5 ng/μL; said E2 is present at a concentration of about 150 ng/μL; or said E3 is present at a concentration of about 5 ng/μL.

14. The method of claim 11, wherein said amount of ubiquitinated p27 formed in the presence of said compound is lower the amount of ubiquitinated p27 formed in the absence of said compound, and said compound is identified as a compound that inhibits the ubiquitination of p27.

15. The method of claim 11, wherein said E1, E2, E3 or Cks1 are recombinantly produced.

16. The method of claim 11, wherein said E1, E2, E3 or Cks1 are purified from a cell extract.

17. The method of claim 11, wherein said ubiquitin is labeled with a label.

18. The method of claim 17, wherein said label is biotin.

19. The method of claim 17, wherein said labeled ubiquitin is visualized using Europium labeled streptavidin.

20. The method of claim 11, wherein the identification occurs in a multi-well plate as part of a high-throughput screen.

21. A method of determining the amount of ubiquitin in a ubiquitinated protein, comprising:
a. labeling said protein with a first label;
b. labeling said ubiquitin with a second label;
c. determining a ratio of said second label to said first label, wherein a higher ratio indicates a greater amount of ubiquitination of said protein.

22. The method of claim 21, wherein said protein is p27.

23. The method of claim 21, wherein said first label and said second label are fluorescent labels.

24. The method of claim 21, wherein said determining a ratio comprises detecting fluorescence from said first label to produce a first fluorescence value; detecting fluorescence from said second label to produce a second fluorescence value; and determining the ratio of said first fluorescence value to said second fluorescence value.

25. The method of claim 21 wherein said second label emits a detectable fluorescence signal when said first fluorescent label is excited.

26. The method of claim 21 wherein said first label and said second label are present on the same ubiquitinated p27.

27. The method of claim 26 wherein said first label emits a detectable fluorescence signal when said second fluorescent label is excited.

28. The method of claim 27 wherein said first label and said second label are present on the same ubiquitinated p27.

29. The method of claim 21, wherein said first label and said second label are suitable for use in a fluorescence resonance energy transfer assay.

30. The method of claim 21, wherein said first label is Europium, Cy5, trisbipyridine europium cryptate, Dylight™ 547, Dylight™ 647, or allophycocyanine (XL665).

31. The method of claim 21, wherein said second label is Europium, Cy5, trisbipyridine europium cryptate, Dylight™ 547, Dylight™ 647, or allophycocyanine (XL665).

32. The method of claim 21, wherein said first label is Europium and said second label is Cy5.

33. The method of claim 32, wherein said Europium is excited by radiation at 340 nm, the fluorescence of said Europium at 620 nm is determined to produce a first fluorescence value, the fluorescence of said Cy5 at 665 nm is determined to produce a second fluorescence value, and a ratio of the second fluorescence value to the first fluorescence value is determined, wherein a higher ratio indicates a greater amount of p27 ubiquitination.

34. A method of identifying an anticancer agent, comprising determining the amount of ubiquitinated p27 formed by combining isolated p27, E1, E2, E3, Cks 1, Cyclin E, Cdk2 and ubiquitin in the presence of said compound and in the absence of said compound; wherein, if the amount of ubiquitinated p27 formed in the presence of said compound differs from the amount of ubiquitinated p27 formed in the absence of said compound, said compound is identified as an anticancer agent.

35. The method of claim 34, wherein said p27 is phosphorylated with Cdk2 and Cyclin E prior to combination with said E1, E2, E3, Cdk2 and ubiquitin.

36. The method of claim 34, wherein said phosphorylated p27 is present at a concentration of about 4 ng/μL; said E1 is present at a concentration of about 5 ng/μL; said E2 is present at a concentration of about 150 ng/μL; or said E3 is present at a concentration of about 7.5 ng/μL.

37. The method of claim 34, wherein said amount of ubiquitinated p27 formed in the presence of said compound is lower the amount of ubiquitinated p27 formed in the absence of said compound.

38. The method of claim 34, wherein said E1, E2, E3 or Cks1 are recombinantly produced.

39. The method of claim 34, wherein said E1, E2, E3, and Cks1 are purified from a cell extract.

40. The method of claim 34, wherein said ubiquitin is labeled with a label.

41. The method of claim 40, wherein said label is biotin.

42. The method of claim 40, wherein said labeled ubiquitin is visualized using Europium labeled streptavidin.

43. The method of claim 34, wherein the identification occurs in a multi-well plate as part of a high-throughput screen.

44. A kit for p27 ubiquitination assay comprising p27; Cdk2/Cyclin E; and a plurality of polypeptides, said plurality of polypeptides together capable of ubiquitinating p27, recombinant cells expressing the polypeptides, or recombinant vectors harboring sequences which encode the polypeptides.

45. The kit of claim 44, wherein p27 and Cdk2/Cyclin E are provided as a complex.

46. The kit of claim 44, wherein said plurality of polypeptides comprises E1, E2, E3, Cks1 and ubiquitin.

47. The kit of claim 46, wherein said ubiquitin is labeled with a label.

48. The kit of claim 47, wherein said label is biotin.

49. The kit of claim 44, which further comprises one or more of plates, optionally coated with Protein A or G; buffers; visualization reagents; or an apparatus or a reagent required for expression and purification of the polypeptides.
